(19) United States
(12) Patent Application Publication
(10) Pub. No.: US 2011/0045091 A1
(43) Pub. Date: Feb. 24, 2011

(54) ANTI-ANGIOGENESIS, ANTICANCER PROLIFERATION PROPERTIES OF LYMPHOCYTIC-DERIVED MICROPARTICLES

(75) Inventors: Pierre Hardy, Verdun (CA); Chun Yang, Brossard (CA)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: CENTRE HOSPITALIER UNIVERSITAIRE SAINTE-JUSTINE

(21) Appl. No.: 12/937,430

(22) PCT Filed: Apr. 9, 2009
(86) PCT No.: PCT/CA09/00457

§ 371 Date: Oct. 12, 2010

Related U.S. Application Data
(60) Provisional application No. 61/044,241, filed on Apr. 11, 2008.

(51) Int. Cl. A61K 9/50; A61K 35/26; A61P 35/00; A61P 27/02; A61P 9/00; C12P 1/00

(52) U.S. Cl. 424/490; 424/578; 435/41

(57) ABSTRACT

Recent studies have demonstrated that lymphocyte-derived microparticles (LMPs) impair endothelial cell function. The present invention concerns the use of LMPs in the regulation of angiogenesis or diseases such as cancer or retinopathy of prematurity (ROP). Having long been considered as cellular debris, microparticles constitute reliable markers of vascular damage. Released into biological fluids, microparticles are involved in the modulation of key functions including immunity, inflammation, vascular remodeling and angiogenesis. The present data demonstrates that LMPs have considerable impact on angiogenesis in vitro and in vivo. In view of this, LMPs may be important contributors to the pathogenesis of diseases that are accompanied by impaired angiogenesis and could thus influence vascular function (microvascular angiogenesis and vasopermeability of ischemic tissue, alerting the body for special attention and the need for emergency repair procedures.

### FIELD OF THE INVENTION

The present invention relates to lymphocytic-derived microparticles (LMPs) and their use for the prevention or treatment of diseases such as oxygen-induced retinopathy, cancer or conditions involving angiogenesis.

### BACKGROUND OF THE INVENTION

Microparticles (MPs) are small membrane vesicles¹ released upon activation or during apoptosis from various cell types, including lymphocytes, platelets and endothelial cells^{2, 3}. Microparticles have been implicated in the pathogenesis of cardiovascular and inflammatory diseases that are associated with vascular damage and impaired angiogenesis. Of relevance, lymphocyte-derived MPs (LMPs) have been detected at elevated levels in atherosclerotic plaques and in patients with myocardial ischemia or preeclampsia². Recent observations have further demonstrated that MPs released from apoptotic lymphocytes or from plasma of diabetic patients induce endothelial dysfunction by modulating nitric oxide pathways⁴.

Angiogenesis is involved in physiological events such as embryonic development and wound healing, as well as in pathological conditions such as tumor growth, diabetic retinopathy, and chronic inflammation⁵. This tightly regulated and complex process involves endothelial cell survival, proliferation, migration, differentiation, and tube formation. It is widely accepted that angiogenesis is determined by a relative balance between pro- and anti-angiogenic factors. Vascular endothelial growth factor (VEGF) is one of the most potent angiogenic factors known and exerts its mitogenic effects primarily through the VEGF receptor type 2 (VEGFR2), which is almost exclusively expressed on endothelial cells. Moreover, VEGFR2 possesses intrinsic tyrosine kinase activity and therefore transduces signals leading to stimulation of mitogen activated protein kinases (MAPK). Nonetheless, angiogenesis is also determined by the presence of angiostatic molecules. CD36 is a potent anti-angiogenic surface receptor that is expressed by microvascular endothelial cells and binds to numerous ligands, including thrombospondin (TSP)-1, an endogenous inhibitor of angiogenesis. Interestingly, a previous study demonstrated that activation of CD36 by TSP-1 down-modulated VEGFR2 expression and p38 MAPK phosphorylation. Then again, increased CD36 expression has been associated with pro-oxidative conditions such as atherosclerosis, inflammation, and ischemia.

Reactive oxygen species (ROS) are involved in the development and progression of various cardiovascular diseases and oxidative stress is considered the central mechanism. Furthermore, oxidative stress is thought to contribute to angiogenesis by mediating endothelial cell proliferation and migration. The major source of ROS in endothelial cells is NADPH oxidase (NOX); increasing NOX-driven ROS stimulates VEGF expression and enhances VEGFR2 autophosphorylation. In this context, LMPs could be one of the key factors linking oxidative stress and angiogenesis.

Previously published studies have documented that microparticles released from platelets (PMPs) induce angiogenesis and stimulate post-ischemic revascularization, whereas endothelial cell derived microparticles (EMPs) suppress angiogenesis by altering the redox balance⁶. Nevertheless, the involvement of LMPs in regulating angiogenesis is yet to be established.

Cell membrane microparticles (MPs) circulate in the blood of healthy donors, and their elevated counts have been documented in various pathologies. Microparticles are phospholipid microvesicles of 0.05 to 1.5 microM in size, containing certain membrane proteins of their parental cells. MPs may facilitate cell-to-cell interactions, induce cell signaling, or even transfer receptors between different cell types. This is important for transfusion medicine because MPs are present in both plasma and cellular blood products. T lymphocyte has crucial roles in shaping cancer development and MPs derived from T lymphocytes have been identified in plasma and demonstrated to induce endothelial dysfunction.

A growing number of serious, debilitating and often fatal diseases are associated with angiogenesis. These diseases are cumulatively called angiogenic diseases.

Additionally, rapid and excessive angiogenesis accompanies the growth of the solid tumors. Many tumors seem to produce factors such as VEGF which increase cell division of vascular endothelial cells and stimulate the migration and organization of endothelial cells into vessels resulting in neovascularization. Since there is no effective treatment available, and since angiogenic diseases present a serious medical problem, there is an ongoing need for new and more efficient antiangiogenic agents. The search for neovascularization inhibitors has been recently vigorously pursued. Despite this, there remains a need for such inhibitors.

The present invention seeks to meet this and related needs.

### SUMMARY OF THE INVENTION

Herein, it is reported for the first time that LMPs significantly inhibit blood vessel formation in the ex vivo aortic ring angiogenesis assay and in vivo corneal neovascularization (CNV) model. Moreover, the current findings suggest that LMPs strongly diminish VEGF induced endothelial cell proliferation and migration by enhancing ROS production primarily from NOX with accompanied increases in CD36 expression and suppression of VEGFR2 signaling.

More specifically, the present invention relates to the successful generation T lymphocyte-derived microparticles (LMPs). LMPs are small vesicles (0.05-1.5 microM) released from the plasma membrane of human lymphoid CEM T cells with actinomycin D stimulation. It was found, for the first time, that LMPs potently inhibited VEGF-induced inflammatory corneal neovascularisation and aortic ring neovessel formation. LMPs dramatically abrogated VEGF-induced endothelial cell proliferation and migration at a final concentration of 5-10 μg/mL and proved to be efficient in vivo by blocking vascular neovascularisation observed in a model of oxygen-induced retinopathy. This finding could be significant for treatments relating to a number of conditions, including retinopathy of prematurity (ROP).

Significantly, it was also found that LMPs effectively inhibit human endothelial cells proliferation by targeting the VEGF signal pathway. VEGFR2 and phosphorylated ERK1/2 were significantly downregulated by LMPs in human endothelial cells, which are the main downstream effectors of the VEGF signalling pathway.

LMPs also have a strong inhibitory effect on the cell proliferation of all the tested cancer cells, such as Hela, Lewis lung cancer cell and Neuroblastoma cell (N2A). It is noteworthy that LMPs have no effect on the normal terminal differentiated neuron cells, which means that LMPs specifically target the highly activated proliferating cells such as tumor cells and the endothelial cells in tumor tissues, and this has been proven in vivo by repressing cancer cell growth and inhibiting angiogenesis in a mouse model implanted with Lewis Lung Carcinoma Primary Tumors. Local and systemic therapy with LMPs causes a dramatic suppression of inflammation-induced or tumor-induced angiogenesis, and it exhibits strong anti-tumor activity. The present findings prove that LMPs are novel inhibitors of angiogenesis useful for treating angiogenesis-related diseases, such as angiogenesis-dependent cancer.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

FIG. 1. LMPs inhibit angiogenesis in the aortic ring assay and in vivo corneal neovascularization model. (A) Representative illustrations of neovessels arising from aortic rings after 2 and 4 days treatment with saline or 30 μg/mL LMPs. (B) Quantitative analysis of the area of neovessel formation in aortic rings (n=8 per group). Scale bar: 200 μm. (C) Mice subjected to inflammation-induced CNV were treated three times daily for 7 days with vehicle or 50 μg/mL LMPs. (D) Quantification of the vascularized corneal area (n=7 per group). Values are means±SE. *p<0.05; ***p<0.001 vs. CTRL.

FIG. 2. LMPs reduce endothelial cell survival and proliferation. (A) HUVEC or (B) HMEC-1 were incubated with the indicated concentrations of LMPs for 24 hours and cell viability was evaluated by MTT assay. (C) HUVEC were treated with or without 10 μg/mL LMPs for 24 hours. Cell proliferation was assessed by [³H]-thymidine DNA incorporation and normalized to control. (D) HUVEC were treated with 10 μg/mL LMPs for different time periods, then apoptotic cells were determined by flow cytometry and expressed as the percentage of apoptotic cells relative to the total number of cells per condition. Values are means±SE of 3-5 individual experiments, each performed in triplicate. **p<0.01, ***p<0.001 vs.CTRL.

FIG. 3. Antioxidants partially restore the LMP-mediated anti-proliferative effects. HUVEC were pretreated for 3 hours with the indicated concentrations of (A) U83836E, U74389G (B) apocynin or (C) diphenyleneiodonium (DPI), after which 10 μg/mL LMPs were added and incubated for an additional 24 hours followed by cell proliferation measurements. Values are means±SE of 3 individual experiments performed in triplicate.*p<0.05, ***p<0.001 vs. LMP treatment.

FIG. 4. LMPs induce ROS production and NADPH-dependent superoxide generation. (A) HUVEC were incubated with 10 μg/mL LMPs in the absence or presence of apocynin (1 mM). Intracellular ROS generation was measured by DCF fluorescence. Data are expressed as relative to control. (B) HUVEC were incubated with 10 μg/mL LMPs for different time points, superoxide anion (O₂⁻) production was measured as lucigenin-enhanced chemiluminescence using NADPH as substrate. Data are expressed as percentage of control. Values are means±SE of 3 individual experiments, each performed in triplicate. *p<0.05 vs. CTRL, #p<0.05 vs. LMPs, ***p<0.001 vs. CTRL.

FIG. 5. LMPs increase p22^{phox}, p47^{phox}, gp91^{phox}, and CD36 expression. (A, C, E,) HUVEC were treated with 7.5 and 15 μg/mL LMPs for 24 hours and p22^{phox}, p47^{phox}, gp91^{phox} expression was detected by Western blot. (B, D, F) The p22^{phox}, p47^{phox} and gp91^{phox} protein levels were normalized to β-actin and the untreated condition was set to equal 100%. (G, H) CD36 protein levels were determined by Western blot in human microvascular endothelial cells treated with 10 and 15 μg/mL LMPs and data was normalized to β-actin. Values are means±SE of 3 experiments. *p<0.05 vs. control.

FIG. 6. LMPs inhibit VEGF-induced cell migration. (A) Migration of HUVEC was induced by 10 ng/mL VEGF in the absence or presence of 10 μg/mL LMPs. Photographs were taken at 48 h and 72 h. Black arrows indicate direction of cell migration. Representative images (4×) are shown from three independent experiments performed in duplicate. (B) Cell migration (72 h) was quantified by MTT assay and is represented as the relative cell migration rate compared to VEGF alone ***p<0.001. (C) LMPs inhibit VEGF-induced cell migration in Boyden chamber assay. HUVEC were incubated with 10 μg/mL LMPs in the absence or presence of 1.5 mM apocynin for 24 hours under induction of VEGF (10 ng/mL). The total number of migrated cells per well were counted and presented as means±SE. ***p<0.001 vs. VEGF and ##p<0.01 vs. VEGF+LMP.

FIG. 7. LMPs downregulate VEGFR2 protein expression and ERK 1/2 phosphorylation. (A) HUVEC were treated with a VEGFR2 polyclonal antibody (1.5 μg/mL) in the absence or presence of LMPs (10 μg/mL) for 24 hours followed by cell proliferation measurements. Relative proliferation rates are presented as means±SE. **p<0.01 vs. CTRL; #p>0.05 vs. LMP or Ab-VEGFR2 group. HUVEC were pre-exposed to 7.5 and 15 μg/mL LMPs for 24 hours, VEGFR2 protein level (B) and phosphorylated, total ERK1/2 (D) were determined by Western blot. (C) VEGFR2 protein levels were normalized to β-actin and presented as relative to control, **p<0.01 vs. CTRL.(E) The level of phosphorylated ERK1/2 was normalized to total ERK1/2 and depicted as relative to control, *p<0.05 vs CTRL.

FIG. 8. Total microparticle levels (A) measured in retinal tissues of rat pups at P14 and P18 in normoxia (white bar) compared to the oxygen-induced retinopathy model (black bar). Lymphocytic microparticle subpopulation was measured in retinal tissues (B) and in plasma from orbital sinus (C) of rat pups at P14. *p<0.05, **p<0.01. The oxygen-induced retinopathy model (OIR) is the rat model of ROP, retinopathy of prematurity.

FIG. 9. LMPs inhibited HREC (human retinal endothelial cells) cell viability and proliferaton. HREC cell viability and proliferation rate were measured by MTT assay (A) and H³-thymidine incorporation assay (B) after HREC were incubated with different concentrations of LMPs for 24 hours. ***P<0.001.

FIG. 10. LMPs inhibited VEGF-induced HREC migration. HREC migration were measured after 48 hours of incubation with 10 ng/mL of VEGF (A) and VEGF+10 μg/mL of LMPs (B). (C) Graph represents the relative amount of migration cells. VEGF group was set as 100%, **P<0.01. Arrows indicate the migration direction of endothelial cells.

FIG. 11. The ERK and Akt protein level was detected by Western blot. Phosphorylated-Akt (A) and phosphorylated-ERK (B) were detected in HREC at indicated time points after incubation with 10 μg/mL of LMPs. The relative protein levels were presented as percentage of control (0 minute).*P<0.05; **P<0.01 vs control.

FIG. 12. Intravitreal injections of LMPs inhibit developmental retinal angiogenesis of newborn rats. (A) LMPs were injected at P2 and P14, and retinal tissues were collected at P6. The surface area of the retina covered by vessels (vascularized area) was measured and quantified as a percentage of the entire retina. (B) LMPs at the concentration 50 μg/ml significantly reduced retinal vasculogenesis by 11% compared to control, **p<0.01.

FIG. 13. LMPs inhibit retinal neovascularization in rat pups that developed an oxygen-induced retinopathy. (A) Angiogenesis in ROP model of rat pups at postnatal day 20 (P20) treated with or without LMPs. One hundred (100) μg/ml of LMPs were intravitreally injected at P0, P3, P6, P9, P12, P15, P18. *p<0.05. (B) Angiogenesis in ROP model of rat at P20. One hundred (100) μg/ml of LMPs were intravitreally injected at P15 and P18. ** p<0.01.

FIG. 14. In vivo modulation of VEGF-induced retinal vascular leakage. Retinal permeability was measured after an intravitreal injection of 50 ng of VEGF with or without 6 μg of LMPs. *P<0.05 vs control.

FIG. 15. Visualization of red fluorescent-labelled LMPs (Dil-LMPs) in rat retina.

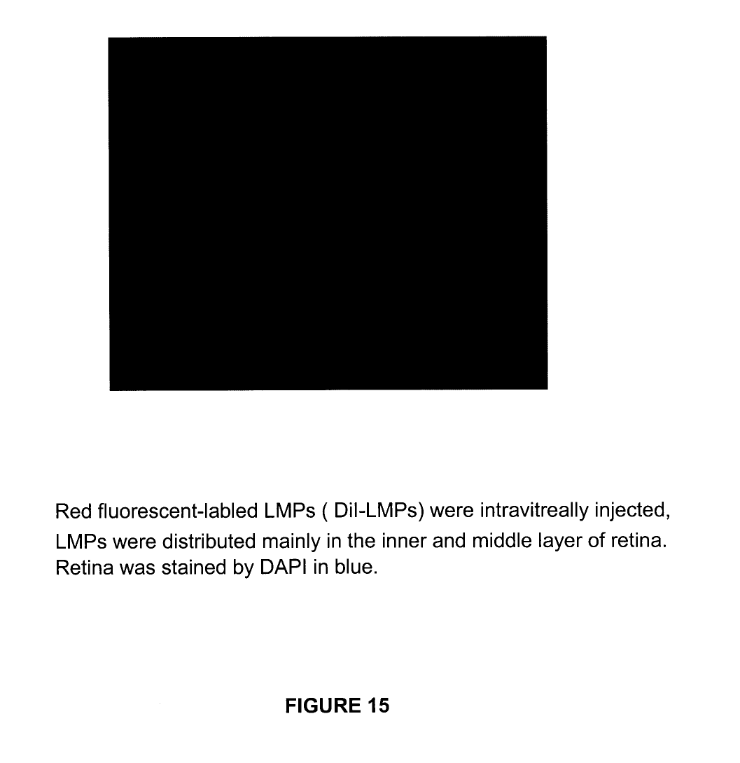

This representative image is showing that LMPs are distributed mainly in the inner and middle layer of retina 24 hours after intravitreal injection with Dil-LMPs. Within the retina cell nucleus are stained by DAPI in blue.

FIG. 16: LMPs generated from three different T lymphocyte sources reduce Lewis Lung carcinoma (LLC) cell viability, but not CEM T cells per se. The LMPs, which were generated from three different T lymphocyte sources, including CEM T, Jurkat and human peripheral T lymphocytes (stimulated by actinomycin D), significantly reduce mouse Lewis lung carcinoma cells (LLC) viability. LLC cells were incubated with 10 μg/mL of each LMPs for 24 hours and cell viability was evaluated by MTT assay (A). LLC cell viability was not affected by the incubation with different amount of CEM T cells for 24 hours. Cell viability was assessed by MTT assay and normalized to control (B).

FIG. 17: LMPs reduce LLC viability and proliferation rate in a dose-dependent manner. LLC cells were incubated with the indicated concentrations of LMPs for 24 hours and cell viability was evaluated by MTT assay (A), and cell proliferation was assessed by [³H]-thymidine DNA incorporation (B).

FIG. 18: LMPs induce LLC apoptosis in a dose-dependent manner. LLC cells were treated with indicated concentrations of LMPs for 24 hours, followed by incubation with reagents from the Vybrant Apoptosis Assay Kit. The apoptotic cells were determined by flow cytometry and expressed as the percentage of apoptotic cells relative to the total number of cells per condition.

FIG. 19: LMPs significantly down-regulate VEGF expression in LLC cells and LLC tumors. After 24 hours incubation with 30 μg/mL of LMPs, the VEGF-A levels in LLC culture medium and cell lysates were measured by ELISA and normalized to protein concentrations and the untreated condition was set to 100%. The VEGF-A protein level was also detected in the LLC primary tumors which were from the treatment of LLC with LMPs in allograft mice. (FIG. 22).

FIG. 20: LMPs reduce microvessel density in LLC tumors. Representative images showing microvessels in the LLC tumor cryosections stained positively with Lectin-TRITC. Upper panel images (A, C) are from control mice receiving 1× PBS intratumor injection; Lower panel images (B, D) are from LMPs treated mice receiving 50 μg of LMPs by intratumoral injection. Microvessel density was determined using Image-Pro imaging software, four different fields were measured and the average values depicted as percentage of the control (E).

FIG. 21: LMPs suppress LLC tumor development in vivo. (A) Representative tumor-bearing C57BL/6 female mice after 10 days of S.C. inoculation with 0.5 million of LLC in PBS or LLC with LMPs (50 μg). (B) LLC primary tumors were weighted and presented as means±SE of 8 mice in each group. Administration of 50 g of LMPs significantly inhibits the tumor development compared to control.

FIG. 22: LMPs inhibit primary LLC tumor progression by intratumoral injection. (A) Representative treated and untreated tumor-bearing C57BL/6 female mice. After 0.5×10⁶ of LLC inoculated s.c. for 7 days, C57BL/6 mice received intratumor injection of either 50 μl of PBS (control group) or 2.5 mg/kg of LMPs every two days for consecutive 4 injections. Primary tumours were collected on day 14 and weighted. (B) LMPs significantly attenuated tumor growth compared to PBS injection group, 5 mice in each group.

FIG. 23: LMPs down-regulate LDLR expression in N2A. N2A cells were treated with 10 ug/mL of LMPs for 24 hours, and their mRNA expression patterns were analyzed using Affymetrix genechip analysis. Three independent cell lots of N2A were used for replicates of each experimental condition. LMPs down-regulate LDLR gene expression in N2A cells by 52%.

FIG. 24: Base on our data, we surmise that LMPs reduction of VEGF/VEGFR and LDLR may result from the LDLR endocytosis. The regulatory proteins, lipids and cholesterol are up taken by tumor cells which highly expressed LDLR and showing high LDLR activities, and these molecules activate certain signalling pathways and consequently down regulate VEGF-A and LDLR expression to interfere the tumor cell growth.

FIG. 25: LMPs have no effect on confluent primary cultured adult rat cortical neuron. The cortical neuron cells were isolated from adult rat and cultured in a confluent condition. The cell viability was analyzed by MTT assay after cells treated with indicated concentrations of LMPs for 24 hours.

FIG. 26: LMPs reduce high proliferating (RGC-5) viability in a dose-dependent manner; whereas they have no effect on the growth of terminal differentiated RGC-5. RGC-5 are immortalized retinal ganglion cells (A), the terminal differentiation of RGC-5 (B) was induced by treatment of proliferating RGC-5 cells with the broad-spectrum protein kinase inhibitor staurosporine (SS) at 1 μM concentration.

FIG. 27: LMPs and CEM T cells have no effect on primary cultured rat neuronal cell viability. T cell 1x: 4.5×10⁴ cells. LMPs have strong effect on highly proliferating cells however they have no effect on normal terminal differentiated cells. These results suggest that antiproliferative effect of LMPs is cell type dependent.

FIG. 28: LMPs reduce the viability of three different neuroblastoma cells. LMPs dose-dependently reduce cell viabilities of mouse neuroblastoma cells (N2A, A) and human neuroblastoma cells (SK-N-MC, B; SH-SY5Y, C).

FIG. 29: LMPs inhibit neuroblastoma cell proliferation and induce human neuroblastoma cells death. (A) Mouse neuroblastoma cells (N2A) were incubated with the indicated concentrations of LMPs for 24 hours and cell proliferation was assessed by [3H]-thymidine DNA incorporation, values were normalized to control. (B) SH-SY5Y cells were treated with 30 μg/mL of LMPs for 6 hours, followed by incubation with reagents from the Vybrant Apoptosis Assay Kit. The apoptotic cells were determined by flow cytometry and expressed as the percentage of apoptotic cells relative to the total number of cells per condition.

FIG. 30: LMPs strongly down-regulate VEGF expression in human neuroblastoma cells. After 24 hours treatment of 30 ug/mL of LMPs, the VEGF levels in SH-SY5Y culture medium and cell lysates were measured by ELISA and normalized to protein concentrations and the untreated condition was set to equal 100%.

FIG. 31: LMPs inhibit neuroblastoma growth through down-regulation of ALK expression. (A) Microarray analysis revealed that ALK gene expression in N2A cells was reduced by 52% after treatment with 10 μg/mL of LMPs for 24 hours. (B) ALK protein level in human neuroblastoma cells (SH-SY5Y) cells was detected by Western blot analysis and the result showed a 48% reduction of ALK expression in the SH-SY5Y cells treated by 30 μg/mL of LMPs for 24 hours.

FIG. 32: LMPs suppress human neuroblastoma tumor growth in vivo. This slide represents respectively, treated and untreated NB tumor-bearing nu/nu nude mice. 20×10⁶ of human NB cells (SH-SY5Y) were inoculated s.c. into nu/nu mice as day 1. LMPs or PBS was injected intratumorly once per day starting on day 12 for 16 days. Tumors were collected on day 28.

FIG. 33: LMPs reduce breast cancer cells viability. The effects of LMPs on four different human breast cell lines were assessed using MTT assay. The cell viabilities of all cell lines were significantly and dose-dependently reduced by LMPs treatment for 24 hours. (A) MCF-7, estrogen-receptor (ER) positive; (B) SK-BR-3, ER negative; (C) M4A4, ER negative; (D) Hs-578, ER negative.

FIG. 34: LMPs derived from three different T lymphocyte sources reduce MCF-7 cell viability and dose-dependently induce breast cancer cells death. (A) All three different LMP sources (10 μg/ml) (derived from CEM T, Jurkat and human peripheral T lymphocytes) significantly reduce MCF-7 cell viabilities after 24 hours treatment. (B) The apoptotic rate of M4A4 cells treated by indicated concentrations of LMPs for 24 hours were analyzed and presented as percentage of total cell numbers.

FIG. 35: LMPs abrogate VEGF expression in human breast cancer cells (M4A4). After 24 hours treatment with 30 ug/mL of LMPs, the VEGF levels in M4A4 culture medium and cell lysates were measured by ELISA and normalized to protein concentrations and the untreated condition was set to equal 100%.

### DESCRIPTION OF THE PREFERRED EMBODIMENTS

Materials and Methods

Compounds and Reagents

Actinomycin D, 3-(4,5-dimethyl thiazol-2yl)-2,5-diphenyl tetrazolium bromide (MTT), Lucigenin (N,N-dimethyl-9,9′biacridinium dinitrate) (Sigma Aldrich); β-actin (Novus Biologicals); Flk-1 (VEGFR2) rabbit polyclonal antibody and horseradish peroxidase linked anti-rabbit IgG, antibodies against gp91^{phox}, p22^{phox} (FL-195), p47^{phox}, ERK1/2, phospho-ERK1/2, TSP-1, and rabbit polyclonal CD36 antibody (Santa Cruz Biotechnology; Santa Cruz, Calif.); U83836E and U74389G (Biomol, PA, USA); [³H]-thymidine (Amersham, Mississauga, Ontario, Canada); hrVEGF and apocynin (Calbiochem, La Jolla, Calif., USA); mitomycin C (Fluka Biochemika); Annexin-V-Cy5 (BD pharmagen, Sandiego, Calif.); vybrant apoptosis assay kit, propidium iodide (PI) and fluorescent microbeads, 1 μM (Molecular Probes, Eugene, Oreg., U.S.A); NADPH (Roche Diagnostics, Laval, QC Canada); diphenyliodonium (DPI) (Calbiochem, La. Jolla, Calif., USA).

Cell Culture

CEM T cells were purchased from ATCC (Manassas, USA) and cultured with X-VIVO medium (Cambrex, Walkersville, Md.). Human umbilical vein endothelial cells (HUVEC) were purchased from Cambrex (Walkersville, Md.) and cultured as recommended. The immortalized human microvascular endothelial cell line-1 (HMEC-1) was kindly supplied by Dr Candal FJ (Centers for Disease Control and Prevention, Atlanta, Ga.). HMEC-1 were grown in Endothelial Basal Medium (Cambrex,Walkersville, Md.) supplemented with 10% fetal bovine serum (Gibco, Gaithersburg, Md., USA), 100 μg/mL streptomycin, 100 U/mL penicillin, 10 ng/mL epidermal growth factor (BD, Oakville, Ontario, Canada) and 1 μg/mL hydrocortisone (Sigma). Mouse Lewis lung carcinoma cells (LLC) (LLC1, CRL-1642) and human breast cancer cell (M4A4) were purchased from ATCC (Manassas, USA) and cultured in Dulbecco's Modified Eagle's Medium (DMEM) (Gibco BRL, Long Island, N.Y.) supplemented with 10% FBS, penicillin at 100 units/mL and streptomycin at 100 μg/mL.

Production of LMPs

LMPs were generated as described.(16) Briefly, CEM T cells were treated with 0.5 μg/mL of actinomycin D for 24 hours, a supernatant was obtained by centrifugation at 750 g for 15 min, then 1500 g for 5 min to remove cells and large debris. MPs from the supernatant were washed after three centrifugation steps (50 min at 12,000 g) and recovered in saline. Washing medium from last supernatant was used as control. LMPs were characterized with annexin V staining by FACS analysis, and gated using 1.0 μM beads in which 97% of MPs (≦1 μM) were annexin-V-Cy5 positive. The concentrations of LMPs were determined by the BioRad protein assay. Using the same protocol, LMPs were also generated from hyperoxia (95% O₂, 36 h) or hypoxia (5% O₂, 36 h) exposed CEM T cells.

Animals

Six week old male C57BL/6 mice purchased from Charles River (St-Constant, Quebec, Canada) were used according to a protocol approved by the Sainte-Justine Research Center Animal Care Committee.

Aortic Ring Angiogenesis Assay The aortic ring assay was performed as described previously.⁷ In brief, 1 mm thoracic aortas were embedded in 3-dimensional growth factor reduced Matrigel (BD Biosciences) and cultured in EGM-2 medium at 37° C. The culture medium was changed on day 3 and the aortic rings were treated with saline or 30 μg/mL of LMPs until day 7. Aortic rings were photographed on days 5 and 7 using a Nikon eclipse TE300 inverted microscope. The angiogenic response was determined by measuring the area of neovessel formation using Image Pro Plus software.

Murine Model of Corneal Neovascularization

Angiogenesis was investigated in vivo using a murine model of corneal neovascularization (CNV) as described previously.⁷ Briefly, each mouse was anesthetized with isoflurane (Abbott, Canada), and topical proparacaine (Alcon, Canada) and 2 μL of 0.15M NaOH were applied to the central cornea. The corneal and limbal epithelium were removed by scraping with a scalpel. Gentamicin sulfate ophthalmic solution (Sabex Inc., Quebec, Canada) was instilled immediately following epithelial denudation. Buprenorphine (0.05 mg/kg; Schering-Plough Ltd) was administered post-operatively for analgesia. Twenty-four hours after corneal injury, mice were randomly divided into two groups that received either saline or 50 μg/mL LMPs. Treatments were administered topically three times daily for 7 days, after which corneas were harvested, flatmounted and immunostained with FITC-conjugated anti-CD31. Images were captured with a Nikon digital camera DXM 1200 using Nikon ACT 1 version 2.62 software. The CNV was quantified in a masked fashion using Adobe Photoshop 7.0 image analysis software. The total corneal surface area was outlined using the innermost vessel of the limbal arcade as the border and the ratio [(neovascularized area/total cornea area)×100] was used to provide a measure of the percentage vascularized cornea.

Cell Viability Assay

Cells at approximately 60% confluence were incubated for 24 hours with vehicle or the indicated concentrations of LMPs. Cell viability was estimated by mitochondrial-dependent reduction of MTT. Essentially, MTT (0.5 mg/mL in PBS [pH 7.4]) was added to the culture medium and incubated at 37° C. for 3 hours, media was aspirated, formazan product solubilized with acidified isopropanol, and the optical density was read at 545 nm with reference wavelength at 690 nm.

[³H]-Thymidine Incorporation Assay

4×10⁴ HUVEC were plated and serum starved for 24 hours. After synchronization, cells were cultured in complete medium with vehicle or 10 μg/mL LMPs for an additional 24 hours. Thereafter, 1 μCi/mL [³H]-thymidine was added to each well, and incubated for 24 hours. [³H]-thymidine DNA incorporation was assayed by scintillation counting.

Apoptosis Assay

HUVEC were treated with or without 10 μg/mL LMPs for 8, 18 and 24 hours, then treated with reagents from the Vybrant Apoptosis Assay Kit (Molecular Probes, Invitrogen) followed by flow cytometry analysis according to the manufacturer's protocol. The rate of apoptosis or necrosis was expressed as the percentage of apoptotic cells relative to the total number of cells per condition.

Measurement of ROS Generation and the NADPH Oxidase Assay

Induction of reactive oxygen species (ROS) was measured using the fluoroprobe DCFDA (Molecular Probes). Endothelial cells were cultured in 24-well plates and treated with LMPs and/or apocynin at indicated concentrations for three hours, or angiotensin (Ang II; 100 nM) for 45 minutes as a positive control. Cells were stained with DCFDA (10 μM) for another 30 minutes. After staining, the extracellular dye was washed twice with 10.0 mM HEPES buffer (pH 7.4), and the fluorescence was measured at an excitation wavelength of 485 nm and an emission wavelength of 535 nm, using a multi-well fluorescent plate reader (Wallac 1420 VICTOR Multilabel Counter).

NADPH oxidase activity was measured by the lucigenin-enhanced chemiluminescence method. Briefly, HUVEC were treated with 10 μg/mL LMPs for different time periods, washed in ice-cold PBS, harvested, and homogenized via sonication (1 second) (Brandson Sonifier 150, USA) in lysis buffer (20 mM KH₂PO4, pH 7.0, 1 mM EGTA, 10mM complete protease inhibitor cocktail). Homogenates were centrifuged at 800×g at 4° C. for 10 min to remove the unbroken cells and debris, and aliquots were used immediately. To initiate the assay, 100-μl aliquots of the homogenates were added to 900 μl of 50 mM phosphate buffer, pH 7.0, containing 1 mM EGTA, 150 mM sucrose, 5 μM lucigenin, and 100 μM NADPH. Photon emission in terms of relative light units was measured in a luminometer every 2 mins for 30 min. There was no measurable activity in the absence of NADPH. Superoxide anion production was expressed as relative chemiluminescence (light) units (RLU)/μg protein. Protein content was determined by the Bio-Rad protein assay.

Western Blot Analysis

Cells were plated at a density of 1×10⁶ cells per 100-mm plate and incubated with 7.5, or 10 and 15 μg/mL LMPs for 24 hours. Soluble proteins were extracted using cell lysis buffer (10 mM Tris-HCl, 1.5 mM MgCl₂, 1 mM DTT, 1 μM pepstatin, 0.75 mM EDTA, 1% (v/v) SDS, 10 mM protease inhibitor cocktail (Roche, pH 7.5). Following centrifugation, the supernatant was collected and total protein concentration was determined (Bio-Rad assay). 25 μg of protein was fractionated by SDS-PAGE. The resolved proteins were transferred onto a PVDF membrane on a semi-dry electrophoretic transfer cell (Bio-Rad) for Western blot analysis. Membranes were blocked, and then incubated overnight at 4° C. with an anti-VEGFR2 polyclonal antibody (1:500 dilution), anti-gp91^{phox} (1:100), anti-p22^{phox} antibody (1:200), anti-p47^{phox} (1:200), phospho-ERK1/2 antibody (1:200), ERK1/2 (1:200), TSP-1 (1:400), and anti-CD36 polyclonal antibody (1:400). After washing, membranes were incubated with a horseradish peroxidase linked anti-rabbit IgG (1:5000) for 1 h at room temperature. β-actin was used as a loading control (1:10000). Proteins were visualized using the ECL Western blotting detection system (Perkin Elmer).

Cell Migration Assay

Two cell migration assays were used to facilitate analysis. Cell migration was first determined using a coverslip border migration assay. Briefly, 0.5×10⁶ HUVEC were seeded onto 12 mm-coverslips in a 24-well plate. Cells were serum starved for 4 hours and proliferation was inhibited by adding 10 μg/mL mitomycin C for 30 minutes. Next, coverslips were carefully removed, washed with fresh media, and transferred into a 12-well plate containing 10 ng/mL VEGF in the presence or absence of 10 μg/mL LMPs. Images were captured between 48 and 72 hours using an Axiovert 200M inverted microscope (Zeiss). At 72 hours, the coverslips were removed and the proportion of migrated cells was quantified by MTT assay.

The Boyden chamber migration assay was also used. A 96-well chemotaxis chamber with five micron polycarbonate filter was purchased from Corning Incoporated, NY. The filter was placed over a bottom chamber containing 10 ng/ml of hrVEGF. 10,000 HUVEC were seeded to each well in the upper chamber. For testing the effects of LMPs and apocynin on the cell migration, HUVEC were incubated with LMPs and/or apocynin in the upper chambers. The assembled chemotaxis chamber was incubated for 24 hours at 37° C. with 5% CO₂ to allow cells to migrate through the filter. Non-migrated cells on the upper surface of the filter were removed by scraping with a wiper tool (Neuro Probe, Inc., Gaithersburg, Md.) and a cotton swab, and the filter was stained with coomassie blue. The total number of migrated cells per well were counted; the assays were performed in quadruplicate.

Determination of Developmental Angiogenesis in Retina of Eyes Injected with LMPs

Newborn rat pups were anesthetized with isoflurane and injected intravitreally on postnatal days 2 and 4 with 5 μl of vehicle (saline) in the right eye or 50 μg/ml LMP in the left eyes [final intraocular concentrations are based on estimated eye volume⁸. Pups were killed at postnatal day 6, and retinal flatmounts obtained. After TRITC-conjugated lectin staining, vascularized area was analyzed using Image-Pro Plus software.

Rat Model of Proliferative Retinopathy (In Vivo)

A well-established rat model of proliferative retinopathy (ROP) will be applied. In order to induce retinal vaso-obliteration, Sprague-Dawley rats and their nursing mothers were exposed to a variable oxygen environment consisting of alternating cycles of 50% oxygen (24 h) and 10% oxygen (24 h) from P1 to P14 (postnatal day 1 to day 14). Animals were subsequently returned to room air for 4 days generating retinal ischemia. The above conditions were achieved using an Oxycycler (model A820CV, BioSpherix Ltd., Redfield, N.Y., USA). A control group of animals were kept in normoxic conditions and analysed simultaneously.

Presence of LMP in Retinal Tissue

Retinal tissue and blood were collected from control (normoxic) and ROP-exposed rat pups at P14 and P18. MP were isolated based on Canault et al⁹. Retinal tissue was isolated, minced in 0.2 μm filtered Dulbecco's modified Eagles medium, and centrifuged at 400 g (15 min) followed by 12500 g (5 min). The supernatant (retinal homogenate) was used for flow cytometry. MPs were also isolated from whole blood drawn from the retroorbital sinus as described by Combes et al¹⁰. Microparticles were extracted by 2 sequential centrifugations for 15 minutes at 1,500 g, followed by 1 minute at 13,000 g. Supernatant containing microparticles was used for flow cytometry after staining with annexin V. Levels of lymphocytic microparticles were determined by double labelling with annexin and CD4.

Effect of LMP in ROP Model

A group of newborn rat pups subjected to the ROP model and a group of control pups in normoxic condition were anesthetized with isoflurane and injected intravitreally on postnatal days P1, P3, P6, P9, P12, P15 with vehicle (saline) in the right eye or LMP (100 μg/ml) in the left eye. Pups were sacrificed on postnatal day 20, and retinal flatmounts obtained. After TRITC-conjugated lectin staining, vascularized area and vascular density were analyzed using Image-Pro Plus software.

Measurement of Vasopermeability

Assessment of VEGF-induced vasopermeability was determined in male Sprague-Dawley rats, weighing ˜300 g¹¹. After induction of generalized anesthesia, the vitreous of one eye was injected with 50 ng recombinant murine VEGF₁₆₄ (R&D Systems Inc., Minneapolis, Minn.) in 5 μl PBS buffer. The contralateral eye, received an equal volume of PBS, or solution of VEGF containing 6 μg LMP. Approximately 24 hours later, Evans blue was injected through a tail vein at a dosage of 45 mg/kg. After the dye had circulated for 90 minutes, the chest cavity was opened, and rats were perfused via the left ventricle with normal saline. Immediately after perfusion, both eyes were enucleated and the retinas were then carefully dissected. After measurement of the retinal wet weight, Evans blue dye was extracted by incubating each retina in formamide for 16 hours at 72° C., and the concentration measured by spectrophotometry (UV-1600PC; Shimadzu, Kyoto, Japan). The concentration of dye in the extracts was calculated from a standard curve of Evans blue in formamide.

Rat Cortical Neuronal Cultures

Rat cortical neuronal (RCN) were isolated from Sprague-Dawley rat cortices with the described method. Briefly, cortices from adult Sprague-Dawley rat embryos were cleaned from their meninges and blood vessels in Krebs-Ringer's bicarbonate buffer containing 0.3% bovine serum albumin (BSA, Gibco BRL). Cortices were then minced and dissociated in the same buffer with 1,800 U/ml trypsin (Sigma) at 37° C. for 20 min. Following the addition of 200 U/ml DNase I (Sigma) and 3,600 U/ml soybean trypsin inhibitor (Sigma) to the suspension, cells were triturated through a 5 ml pipet. After the tissue was allowed to settle for 5-10 min, the supernatant was collected, and the remaining tissue pellet was retriturated. The combined supernatants were then centrifuged through a 4% BSA layer and the cell pellet was resuspended in neuronal seeding medium, which consisted of neurobasal medium (Gibco), supplemented with 1.1% 100× antibiotic-antimycotic solution (Biofluids), 25 uM Naglutamate, 0.5 mM L-glutamine, and 2% B27 Supplement (Gibco). Cells were seeded at a density of 1.5×10⁵ cells onto each well of 24-well tissue culture plates (Corning) pre-coated with poly-D-lysine (70-150 kD, Sigma). On the following day, cells were treated with different concentrations of LMPs for 24 hours and cell viability was analyzed by MTT assay.

Animal Model of Lewis Lung Carcinoma (LLC) and LMPs

Six week old female C57BL/6 mice purchased from Charles River (St-Constant, Quebec, Canada) were used according to a protocol approved by the Sainte-Justine Research Center Animal Care Committee. Mice were housed in a room maintained at 25±1° C. with 55% relative humidity and given food and water ad libitum. One week later, mice were anesthetized and each was inoculated with 0.5×10⁶ LLC cells by subcutaneous (s.c.) injection in 50 μl PBS on the right flank. Seven days after LLC inoculation, mice were randomly grouped and each mouse was given an intravenous (i.v.) or intratumoral injection of LMPs at 2.5 mg/kg in 50 μl of PBS every two days for consecutive 4 injections. Meanwhile control mice were administered 50 μl of PBS. Mice were observed at least 4 times weekly for signs of toxicity (lethargy, bloating, and ruffling of fur) during and after each treatment. All mice were killed 15 days after LLC inoculation and locally growing tumors were separated from skin and muscles and weighed.

Xenografting and LMPs Treatment

The nu/nu nude mice (Charles River Laboratories International, Inc.) were used for xenografting at the age of 5-7 weeks. The mice were housed in sterile enclosures under specific virus and antigen-free conditions. They are fed ad libitum. The weight and general appearance of the animals were recorded every other day throughout the experiments. The human neuroblastoma cells (SH-SY5Y, 20×10⁶ cells in 0.1 ml of PBS) were implanted s.c. in the right flank using a 23 G needle. Tumour volume measurements were started when the tumour become palpable (˜100 mm³). LMPs treatment began on day 12 once per day for 16 days. For weighing, measurement of tumour volume, and drug injection, the animals will be anesthetized with isofluoran.

Statistical Analysis

All experiments were repeated at least three times and values are presented as means±SEM. Data were analyzed by one-way ANOVA followed by post-hoc Bonferroni tests for comparison among means. Statistical significance was set at p<0.05.

Results

LMPs Suppress Aortic Ring Angiogenesis and In Vivo Corneal Neovascularization

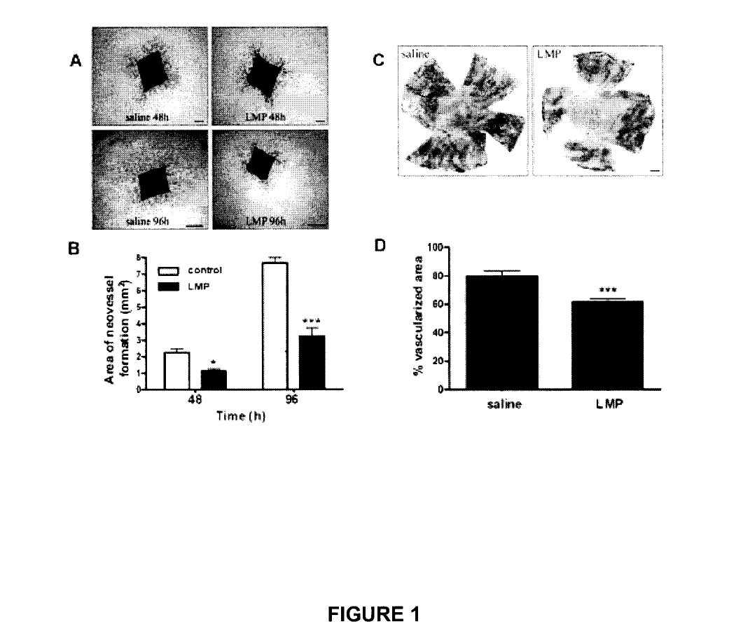

The first objective was to determine whether LMPs affect vessel development. For this purpose, the aortic ring angiogenesis assay was used as well as a pathophysiologically relevant CNV model that is largely driven by VEGF. Incubation of aortic rings with saline or 30 μg/mL LMPs for 48 and 96 hours significantly reduced neovessel formation by 50% (2.2±0.2 mm² vs. 1.1±0.1 mm²; p<0.05) and 58% (7.7±0.3 mm² vs. 3.2±0.5 mm²; p<0.001) (FIGS. 1A, B) respectively. Having established that LMPs inhibit ex vivo angiogenesis, the significance of this in vivo was determined by treating mice subjected to CNV with saline or 50 μg/mL LMPs three times daily for 7 days. Compared with saline treatment, LMPs caused a 23% reduction in CNV (80.0±3.6% vs. 61.6±2.3%; p<0.001; FIGS. 1C, D).

LMPs Inhibit Human Endothelial Cell Survival and Proliferation

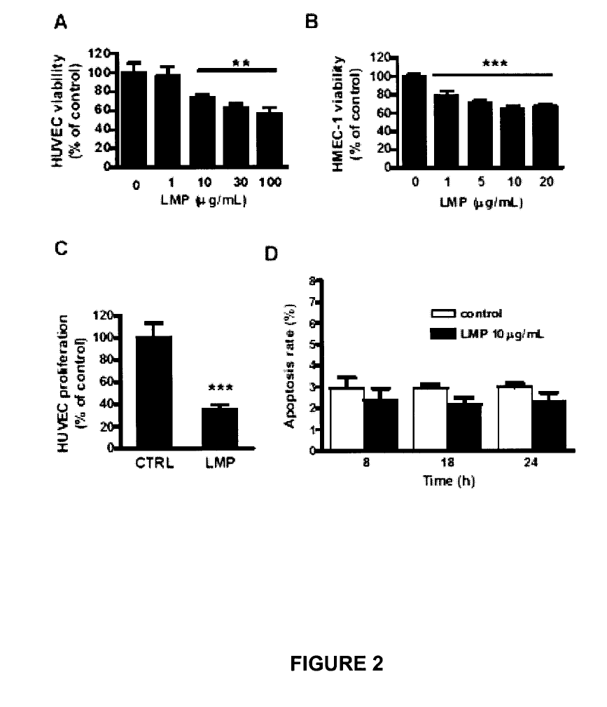

Cell survival and proliferation are critical steps during angiogenesis. To determine the effect of LMPs on vascular cell survival, HUVEC and HMEC-1 were exposed to different concentrations of LMPs and their viability was assessed by MTT assay. LMPs significantly diminished cell viability in both cell types in a concentration dependent manner (FIGS. 2A-B). In order to determine whether the effect of LMPs on cell proliferation is stimulus-dependent, LMPs were generated from hyperoxia or hypoxia exposure. LMPs produced under both hyperoxic and hypoxic conditions potently suppressed HUVEC proliferation (45.8±1.4 and 50.8±2.3, respectively; p<0.001 vs. control) to a comparable degree as actinomycin D-derived LMPs (49.0±0.8; p<0.001 vs. control). This indicates that the effects of LMPs are not stimulus-dependent.

The observed reduction in cell survival could be caused by decreased cell proliferation or increased apoptosis or necrosis. [³H]-thymidine DNA incorporation was applied and LMPs (10 μg/mL) reduced HUVEC proliferation by 60% (p<0.001) (FIG. 2C). To ascertain whether LMPs were inducing apoptosis or necrosis, both LMPs treated and control HUVEC were double labelled with FITC-conjugated annexin-V and PI; however, induction of apoptosis or necrosis was not observed under all test conditions (P>0.05) (FIG. 2D).

Antioxidants Partially Block the Anti-Proliferative Effects of LMPs

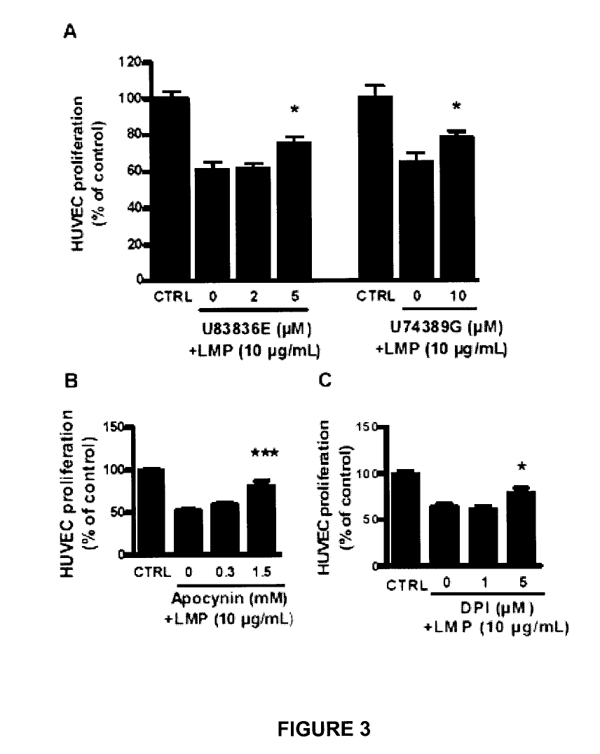

Previous studies have shown that EMPs increase superoxide production and lead to impairment of angiogenic pattern¹². Moreover, NOX, a major source of superoxide free radicals, was highly expressed by endothelial cells. It was therefore postulated that LMPs were exerting their anti-angiogenic properties via oxidative stress mechanisms. To address this hypothesis, utilization was made of two well known lipid peroxidation inhibitors, namely U83836E and U74389G, were tested for their ability to attenuate the anti-proliferative effects of LMPs. U83836E and U74389G at 5 and 10 μM concentrations, respectively, led to a partial but statistically significant increase in cell proliferation compared to LMPs treatment alone (p<0.05) (FIG. 3A). Additionally, pre-treatment of HUVEC with two specific NOX inhibitors, apocynin (1.5 mM) and diphenyleneiodonium (DPI; 5 μM), significantly abrogated the LMP-induced anti-proliferative effects (*p<0.05 and ***p<0.001 respectively; FIGS. 3B-C).

LMPs Increase ROS and NOX Activity

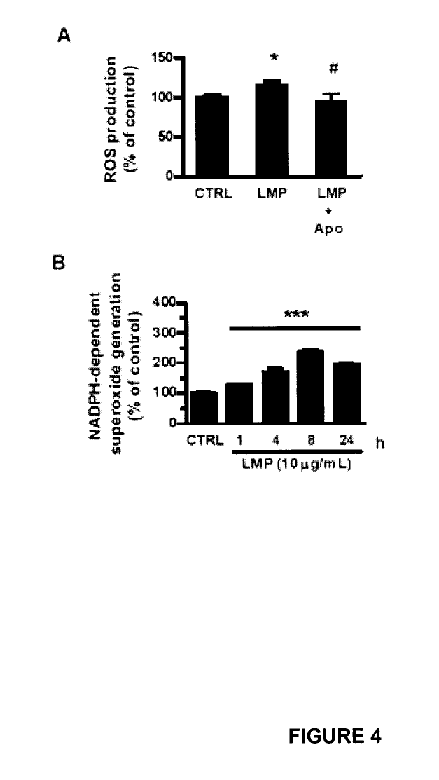

Having demonstrated the important role of oxidative stress in LMP-mediated activities, the effects of LMPs on ROS generation were investigated next. The latter was determined by measurement of intracellular ROS levels using DCF fluorescence following a 3 hour pretreatment with 10 μg/mL LMPs. As shown in FIG. 4A, compared to control, LMPs significantly increased ROS production as indicated by a rise in the DCF signal (p<0.05). Moreover, LMP-induced ROS generation was significantly attenuated by pretreatment with apocynin (1.5 mM; p<0.05).

Because the superoxide-generating NADPH oxidase has been described to largely contribute to ROS formation in endothelial cells, the effect of LMPs on superoxide generation from this enzyme was studied. Superoxide anion production was measured in LMP-treated HUVECs as lucigenin-enhanced chemiluminescence using NADPH as the substrate. As indicated in FIG. 4B, LMPs increased the rate of superoxide formation after 1 hour incubation and reached a peak after 8 hours (P<0.001).

LMPs Induce Protein Levels of p22^{phox}, p47^{phox}, gp91^{phox}, and the CD36 Scavenger Receptor

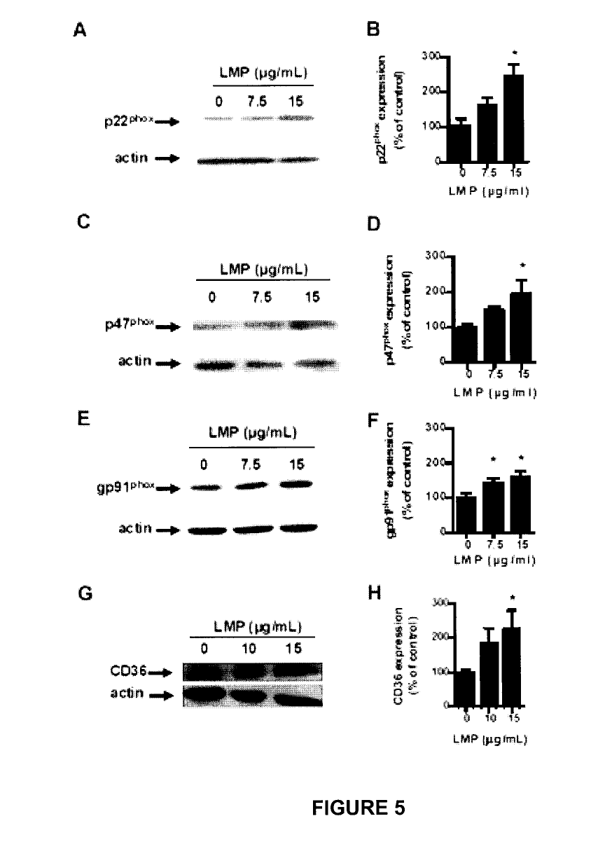

Owing to the ability of LMPs to induce NOX activity, their effect on the expression of p22^{phox}, p47^{phox} and gp91^{phox}, which are critical subunits of NADPH oxidase was determined. Indeed, LMPs strongly upregulated p22^{phox}, p47^{phox} and gp91^{phox} protein expression in a concentration-dependent fashion (P<0.05) (FIGS. 5A-F). Although LMPs demonstrated very low level expression of p22^{phox}, p47^{phox} and gp91^{phox} were undetected.

The CD36 scavenger receptor and its endogenous ligand TSP-1 are potent inhibitors of in vitro and in vivo angiogenesis⁷, whose expression are potentiated in pro-oxidative environments as well as by NADPH oxidase activation. In this context, human microvascular endothelial cells treated with 10 and 15 μg/mL LMPs dose-dependently augmented CD36 protein levels by 1.9 and 2.3 fold, respectively (FIGS. 5G,H), whereas expression of TSP-1 was not significantly changed. Moreover, TSP-1 was not detected in LMPs per se, which is in agreement with the published results from the proteomic analysis of LMPs.

LMPs Mediated Anti-Migratory Effects are Reversed by NOX Inhibitors

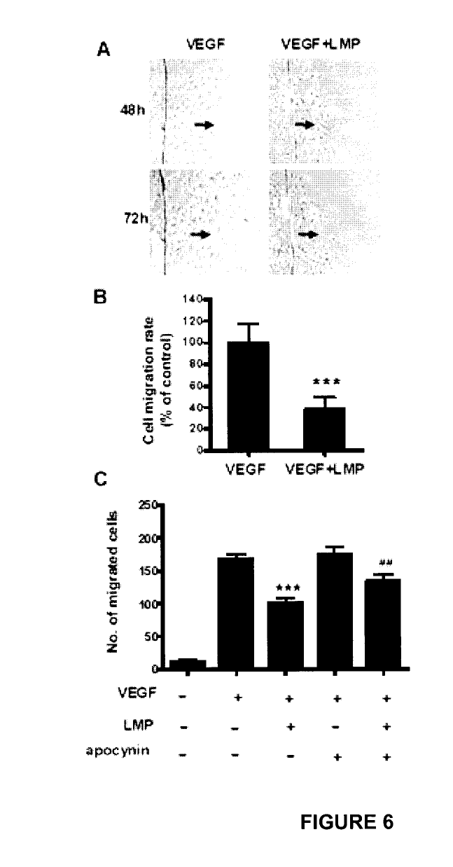

Because cell migration plays a pivotal role in angiogenesis, the effect of LMPs on VEGF-induced cell migration was considered. HUVECs were plated onto coverslips and exposed to 10 ng/mL VEGF with or without LMPs. Cell migration was substantially decreased by 58% after 72 hours of LMPs treatment (p<0.001; FIGS. 6A, B).

Cell migration was also evaluated using the modified Boyden chamber assay. LMPs strongly inhibited VEGF-induced cell migration by 40% (p<0.001; FIG. 6C) and apocynin (1.5 mM) was able to partially rescue LMPs mediated anti-migratory effects (p<0.01 vs. LMP; FIG. 6C).

LMPs Reduce VEGFR2 Protein and Phospho-ERK Levels

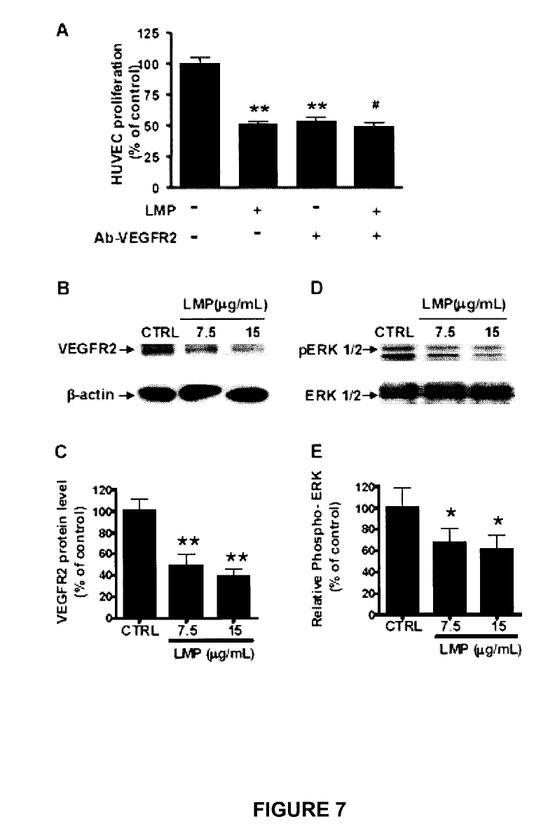

Having observed that LMPs induced CD36 expression, it was surmised that LMPs were further suppressing angiogenesis by antagonizing the VEGF signaling pathway. This hypothesis is corroborated by evidence that activation of CD36 leads to suppression of VEGF-induced VEGFR2 phosphorylation. Accordingly, HUVEC proliferation was assessed following pre-incubation with 1.5 μg/mL anti-VEGFR2 antibody in the presence or absence of LMPs (10 μg/mL). As expected, the anti-VEGFR2 antibody alone strongly decreased cell proliferation (p<0.01); however, co-treatment with the anti-VEGFR2 antibody and LMPs did not result in a synergistic reduction of cell proliferation (#p>0.05, compared to Ab-VEGFR2 group FIG. 7A). Consistent with this data, Western blot analysis of HUVEC treated with 7.5 and 15 μg/mL LMPs, caused a dose-dependent downregulation of VEGFR2 protein expression by 50% and 65%, respectively, vs. control (**p<0.01; FIGS. 7B, C). Phospho-ERK1/2 levels were also significantly inhibited by 35% (*p<0.05; FIGS. 7D, E)

Microparticles (MPs) are known to contribute to the pathogenesis of cardiovascular diseases, including inflammation and vascular dysfunction. Another important action of MPs in the vascular system is their ability to modulate angiogenesis¹³. Nevertheless, despite the escalation in MPs research, very little is known regarding the role of T lymphocyte-derived microparticles (LMPs) in regulating angiogenesis. The present experimental findings demonstrate that LMPs inhibit angiogenesis in vivo and in vitro by suppressing vascular cell survival, proliferation and migration. Significantly, the data demonstrate that LMPs induce ROS production via NOX activation while antioxidants and NOX inhibitors attenuate the anti-angiogenic effects of LMPs. Furthermore, through CD36 induction and VEGFR2 and phospho-ERK1/2 down regulation, evidence is provided to the effect that LMPs interfere with the VEGF signalling pathway. Taken together, these results strongly support a role for LMPs in regulating angiogenesis during pathological conditions.

MPs are released from the plasma membrane during cell activation by apoptosis, shear stress, or agonists. The present studies, MPs were obtained by apoptosis from T lymphocytes treated with actinomycin D. Moreover, the characteristics of MPs appear to depend on the mechanism of stimulation and the activation status of the cell from which they originate^{6, 12}. This is clearly highlighted by the reported effects of MPs on angiogenesis. For example, although it is shown that LMPs possess anti-angiogenic properties, others have shown that MPs from endothelial cells inhibit, whereas platelet-derived MPs promote angiogenesis^{6, 12}. The anti-angiogenic effects of LMPs seem to occur as a result of decreased cell proliferation rather than increased cell apoptosis or necrosis (FIG. 2). This is in agreement with observations by Andriantsitohaina's group who showed that pathophysiological levels of LMPs failed to induce endothelial cell apoptosis¹⁴.

It has been documented that oxidative stress is one of the central mechanisms responsible for endothelial cell dysfunction. The major sources of ROS in endothelial cells are endothelial nitric oxide synthase (eNOS) and NOX. In line with this, there is a general consensus that nitric oxide (NO) inhibits both vascular smooth muscle and endothelial cell proliferation. In this study, nitrite levels were unchanged by LMP treatment and eNOS blockers did not prevent the anti-proliferative effects of LMPs (data not shown). Conversely, LMPs increased ROS levels and NOX activity (FIG. 4) as well as upregulated expression of the gp91^{phox}, p22^{phox} and p47^{phox} NOX subunits (FIG. 5). Consistent with this, inhibition of NOX partly abrogated the inhibitory effects of LMPs on both cell proliferation and migration (FIG. 6C). Collectively, these results support that the NOX, and not the eNOS-NO signaling pathway, is involved in generating ROS that mediate the angiostatic effects of LMPs.

One of the detrimental consequences of oxidative stress is peroxidation of membrane lipids. Lipid peroxidation induces site-specific changes in the organization of the phospholipid bilayer thus leading to cellular dysfunction. The lipid peroxidation inhibitors, U74389G and U83836E, are lipophilic steroid compounds that intercalate into biological membranes, thus enhancing their stability in the event of oxidative stress. In this study, both compounds partially attenuated the anti-proliferative effects of LMPs (FIG. 3A), thus suggesting that LMPs' angiostatic activities also involve increased lipid peroxidation.

Several studies have demonstrated that oxidative stress stimulates CD36 expression and that antioxidants attenuate its expression and function.¹⁵ It was therefore intriguing to observe that LMPs upregulated CD36 expression, which is consistent with the pro-oxidant actions of LMPs (FIGS. 3, 4, 5). However, it is presumed that the LMP-mediated upregulation of CD36 is TSP-1 independent since LMPs had no significant effect on TSP-1 expression. Moreover, because CD36 is a well established anti-angiogenic receptor, it is tempting to speculate that the generation of ROS by LMPs occurs upstream of the induction of CD36 with subsequent suppression of the VEGF/VEGFR2 signaling pathway, as has been proposed here and by others⁷.

It is well known that VEGF plays a pivotal role in developmental and pathological angiogenesis. VEGF stimulates angiogenesis through VEGFR2 (KDR/Flk-1), which is expressed mainly on endothelial cells.¹⁶ In the present study, several lines of evidence supported the hypothesis that LMPs antagonized the VEGF/VEGFR2 pathway. First, LMPs were shown to potently inhibit VEGF-induced inflammatory corneal neovascularization (FIGS. 1C, D). Secondly, VEGF-induced endothelial cell migration was dramatically reduced by LMPs (FIG. 6). Thirdly, inhibition of VEGFR2 activity had no synergistic effect on the anti-proliferative effects of LMPs, suggesting that both VEGFR2 and LMPs signal via the same pathway (FIG. 7A). Finally, it was shown that LMPs significantly downregulated VEGFR2 and phosphorylated ERK1/2 expression (the main downstream effector of the VEGF signaling pathway) (FIG. 7), while increasing CD36 protein levels (FIGS. 5G, H), a known negative regulator of this pathway.

In conclusion, the studies described herein provide evidence for the first time that MPs from T cells inhibit angiogenesis in vivo and in vitro. It was demonstrated that LMPs impair vascular cell survival, proliferation, and migration. The present data also suggests that LMPs regulate angiogenesis by acting through the NAD(P)H oxidase and VEGFR2 pathways. Given the pivotal role of the VEGF/VEGFR2 signaling pathway in angiogenesis, understanding the mechanisms of how LMPs interrupt VEGFR2 signaling could provide attractive therapeutic strategies aimed at reducing the deleterious effects of MPs on the vascular system.

### Example 1

Anti-Angiogenic Properties of Lymphocyte Microparticles in Oxygen-Induced Retinopathy

Retinopathy of prematurity (ROP) is a potentially blinding eye disorder that primarily affects premature infants weighing about 2¾ pounds (1250 grams) or less that are born before 31 weeks of gestation. The smaller a baby is at birth, the more likely that baby is to develop ROP. This disorder, which usually develops in both eyes, is one of the most common causes of visual loss in childhood and can lead to lifelong vision impairment and blindness.

Today, with advances in neonatal care, smaller and more premature infants are being saved. These infants are at a much higher risk for ROP.

Not all babies who are premature develop ROP. There are approximately 3.9 million infants born in the U.S. each year; of those, about 28,000 weigh 2¾ pounds or less. About 14,000-16,000 of these infants are affected by some degree of ROP. The disease improves and leaves no permanent damage in milder cases of ROP. About 90 percent of all infants with ROP are in the milder category and do not need treatment. However, infants with more severe disease can develop impaired vision or even blindness. About 1,100-1,500 infants annually develop ROP that is severe enough to require medical treatment. About 400-600 infants each year in the US become legally blind from ROP.

ROP occurs when abnormal blood vessels grow and spread throughout the retina, the tissue that lines the back of the eye. These abnormal blood vessels are fragile and can leak, scarring the retina and pulling it out of position. This causes a retinal detachment. Retinal detachment is the main cause of visual impairment and blindness in ROP.

LMPs can be considered a hallmark of stress-injured or dying lymphocytic cells and may be recognized in the future as a marker of lymphocytic dysfunction. Alterations in the retinal blood barrier by disorganizing cell-cell junctions, promote shedding of LMPs. However, MPs can no longer be described as passive bystanders awaiting removal by professional phagocytes, as was the previous misconception. It has been reported that in vitro LMPs, at concentrations that can be reached in circulating blood under immunological dysfunction (e.g., HIV), impair endothelium dependent relaxation in conductance and small resistance arteries in response to agonists and shear stress, respectively. Also, LMPs can affect vascular contraction by acting directly on smooth muscle cells.

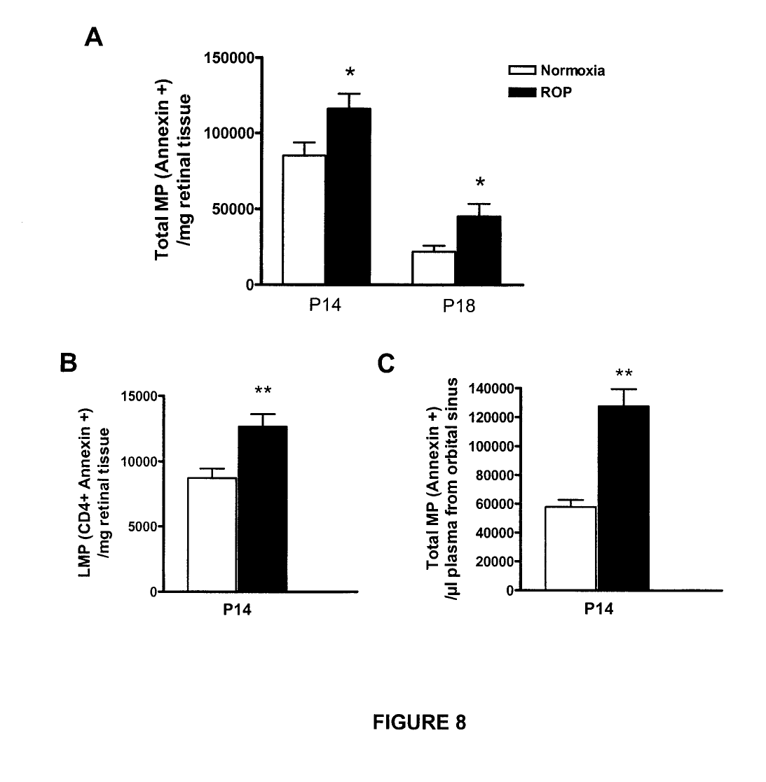

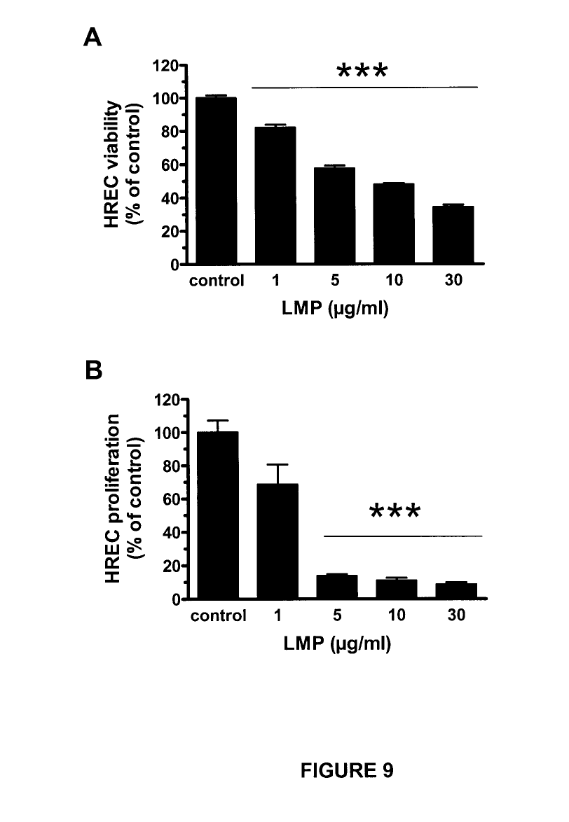

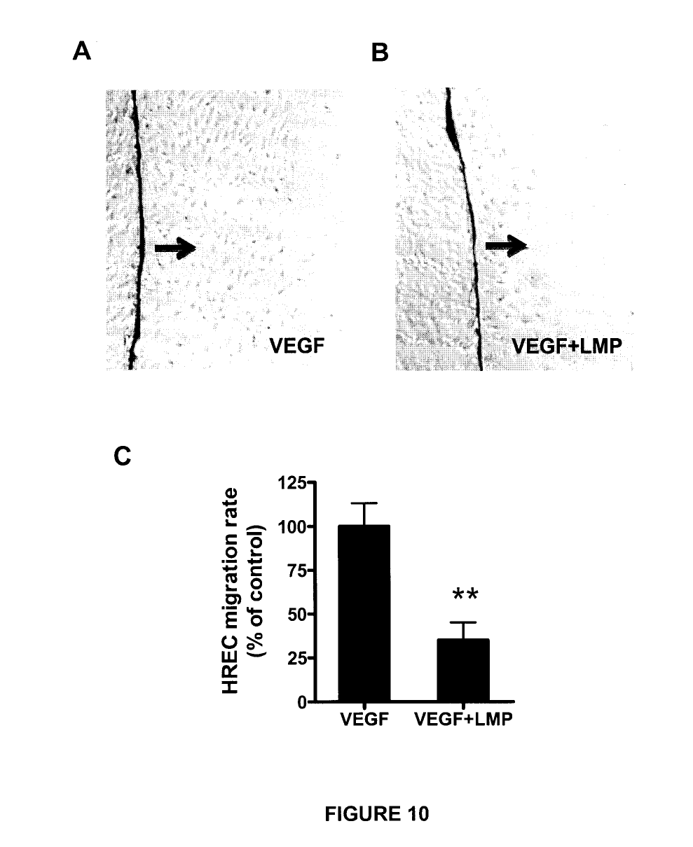

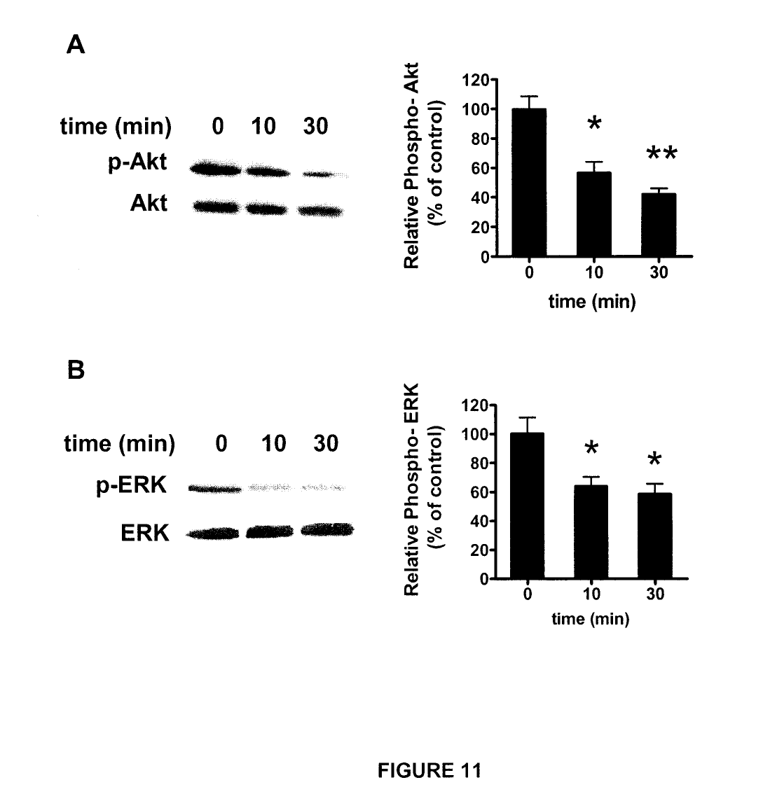

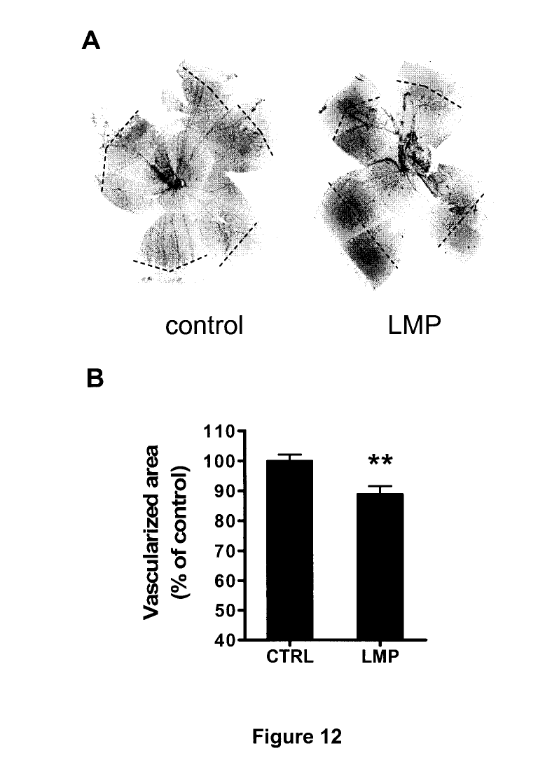

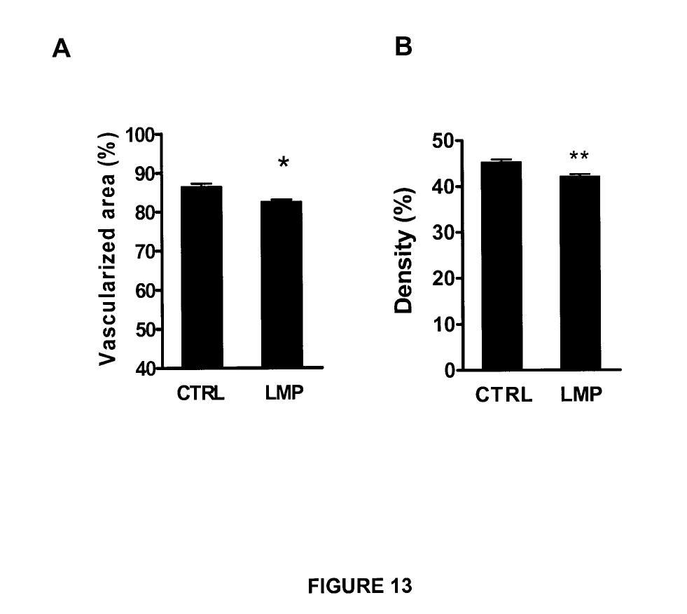

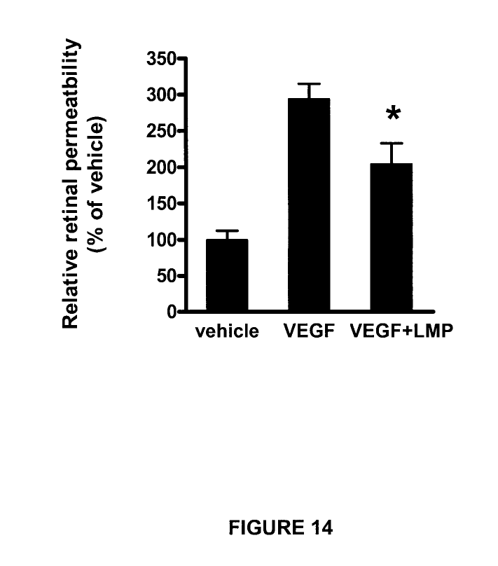

Summary of Findings
LMPs are highly generated in the retinal circulation and exist in the retinal tissue during oxygen-induced retinopathy conditions (FIG. 8).
LMPs exert their antiangiogenic effects primarily through VEGFR-2 and by suppression of its downstream signaling effectors and cascades including Akt and the PKC-ERK1/2 pathway (FIG. 11).
Data indicate that hyperoxia-induced LMPs generated from an in vivo model of retinopathy significantly reduced developmental retinal angiogenesis (FIG. 12).
In the rat model of angiogenesis retinopathy, a significant reduction in retinal neovascularization was observed following LMPs administration (FIG. 13). Because ischemia-induced VEGF is a primary stimulus in this animal model, the present results point towards an anti-proliferative and anti-migration role for LMPs in HREC (FIGS. 9 and 10). Such a suppression of retinal neovascularization in vivo corroborates the hypothesis for LMP-mediated VEGF inhibition.
In the leakage animal model, LMPs significantly reduced VEGF-induced retinal vascular leakage (FIG. 14). This suggest that LMPs interfere with VEGFR-2-mediated PKC activation. Because PKC-β, one of the PKC isozymes, has been postulated as a therapeutic target for VEGF-dependent events in diabetic retinopathy, this result suggest a potent inhibitory action of LMPs in both VEGF and VEGF-dependent PKC signaling in the retina.

The above findings provide a novel therapeutic avenue for the use of LMPs in treating VEGF-based ocular neovascular and vasopermeability conditions including diabetic retinopathy.

LMPs Inhibit Cancer Cell Growth

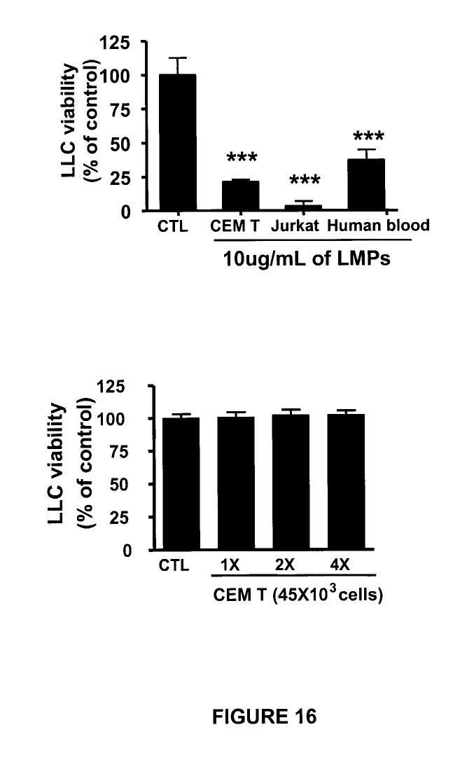

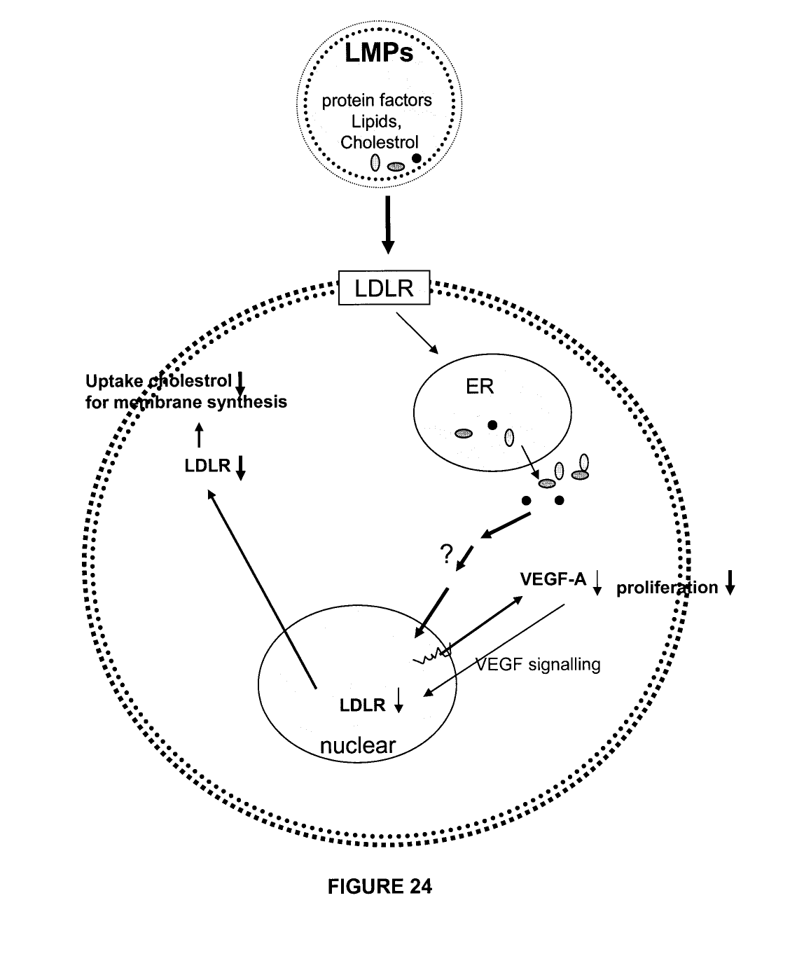

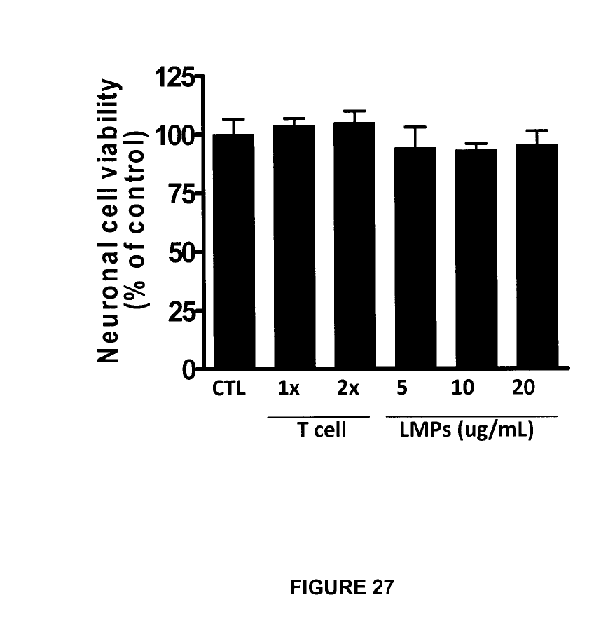

FIGS. 16-35 demonstrate the effects of LMPs on cancer cell survival and growth. The results of these figures may be briefly summarized by the following:

1) LMPs inhibit the growth of several tumor cell lines in vitro.

2) LMPs have no effect on terminal differentiated neuron cell death.

3) LMPs modulate gene expression in neuroblastoma cells (N2A), downregulate VEGFA and several oncogenes, and upregulate tumor supressors.

4) Effect of LMPs on tumor growth in viva

The following Examples will deal more particularly with three different cancer types: Lung Carcinoma (Example 2), Neuroblastoma (Example 3) and Breast Cancer (Example 4).

### Example 2

The Anticancer Effect of LMPs on Lung Carcinoma

Introduction:

Lung cancer is the most common cancer worldwide, and is also the leading cause of cancer death in both men and women in the North American. Angiogenesis, the growth of new vessels from preexisting vessels, is a fundamental step in tumor growth and progression. Vascular endothelial growth factor (VEGF) is a key angiogenic factor implicated in tumor blood vessel formation and permeability. Overexpression of VEGF has been observed in a variety of cancers and has been associated with a worse relapse-free and overall survival.

Mammalian cells obtain the cholesterol necessary for the synthesis of membranes, and rely predominantly on the uptake of lipoprotein derived cholesterol via low density lipoprotein receptor (LDLR) for their cholesterol needs. The LDLR family is a group of receptors that mediate endocytosis leading to lysosomal degradation of an enormous repertoire of ligands. It has been reported that some malignant cell lines have higher LDLR activity than the corresponding normal cells. LDL uptake has been shown to be higher in lung tumour tissue than in the corresponding normal tissue. It is likely that tumour cells need a very high level of cholesterol during their growth phase. This cholesterol might be used for membrane synthesis and could be related to cell growth.

LMPs can function as subcellular vectors to deliver proteins and lipids into target cells and alter the phenotypic properties of the target cells as well as induce a variety of functional changes. Base on the strong antiangiogenic property, we assume that LMPs play an important role in the modulation of lung carcinoma tumor growth.

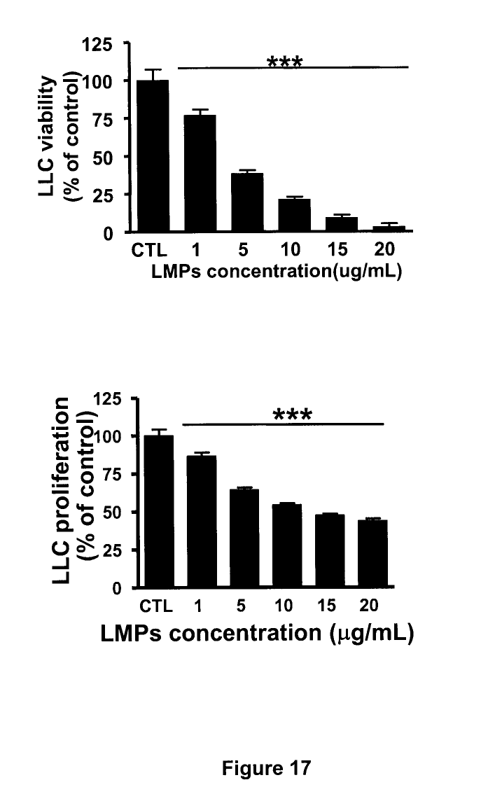

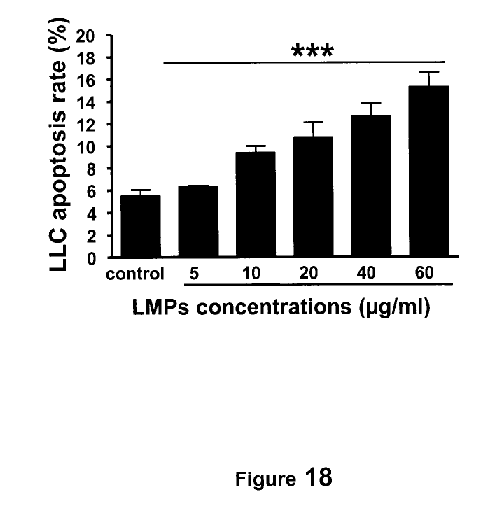

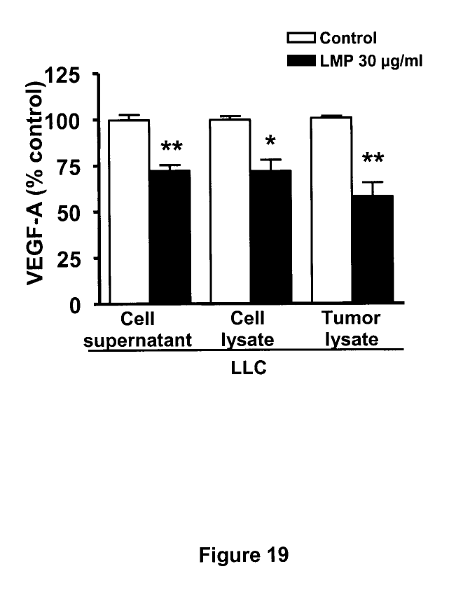

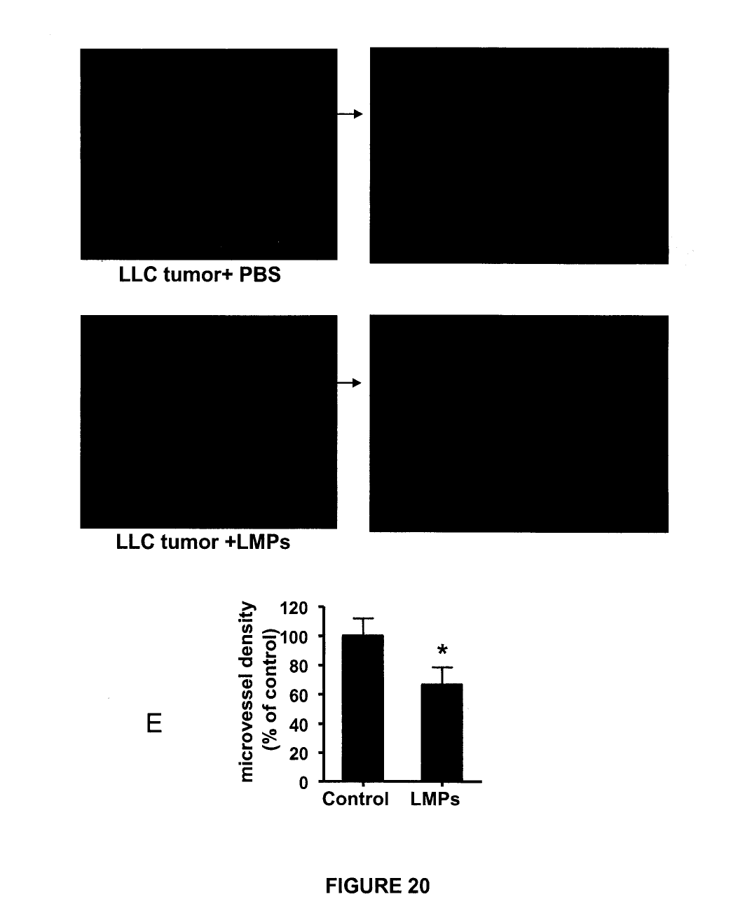

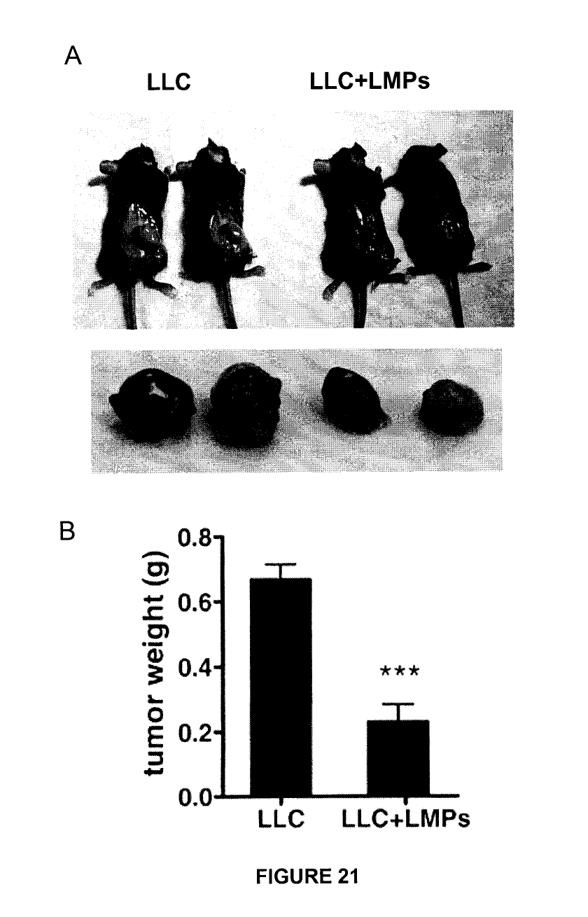

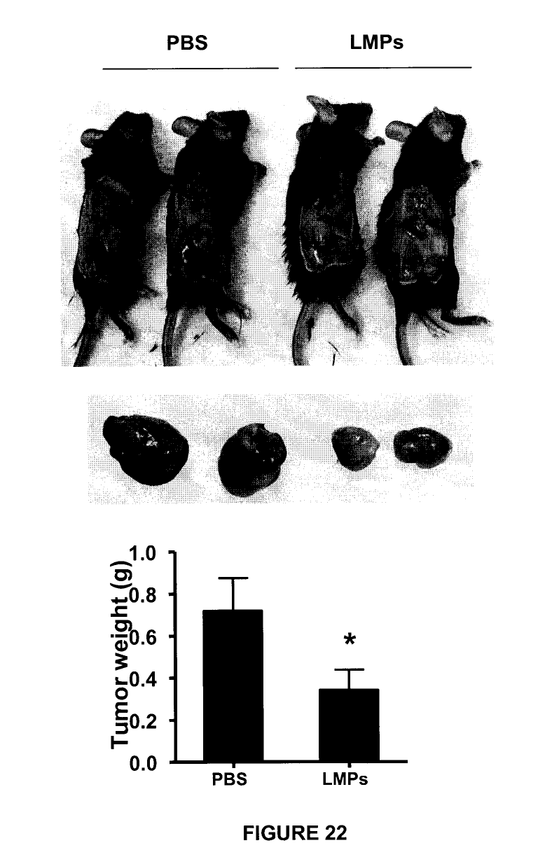

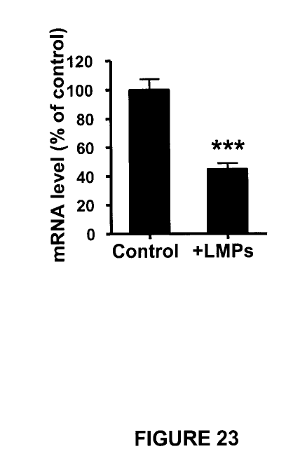

FIGS. 16-23 provide evidence that LMPs are effective in reducing Lewis lung carcinoma (LLC) cell viability. Specifically, the results show the following:
LMPs generated from three different T lymphocyte sources reduce LLC viability, but not CEM T cells per se (FIG. 16).
LMPs reduce LLC viability and proliferation rate in a dose-dependent manner (FIG. 17).
LMPs induce LLC apoptosis in a dose-dependent manner (FIG. 18).
LMPs down-regulate VEGF-A expression in LLC cells and tumors (FIG. 19).
LMPs reduce microvessel density in LLC tumors (FIG. 20).
LMPs suppress LLC tumor development and progression in vivo (FIGS. 21 and 22).
LMPs down-regulate LDLR expression in LLC. (FIG. 23).

Based on this data, it is surmised that the LMPs-relatd reduction of VEGF/VEGFR and LDLR may result from the LDLR mediates endocytosis. The regulatory proteins, lipids and cholesterol were uptaken by tumor cells which highly expressed LDLR and showed high LDLR activities. Proteins, lipids and cholesterol activate certain signalling pathways and consequently may down-regulate VEGF-A and LDLR expression to interfere with the tumor cell growth.

### Example 3

The Anticancer Effect of LMPs on Neuroblastoma

Introduction:

Neuroblastoma (NB) is the most common solid cancer of early childhood, and accounts for 15% of all cancer deaths in children. Despite significant advances in the past three decades, high risk NB has remained an enigmatic challenge to clinical and basic scientists. Anaplastic lymphoma kinase (ALK), a receptor tyrosine kinase, was recently identified as a familial NB predisposition gene. Somatic point mutations in the ALK gene have been found in sporadic cases of NB. These mutations lead to ALK kinase activation, cell transformation and tumorigenicity in vivo¹⁷. Like most human cancers, vigorous neovascularisation is one of the prominent characteristics of high-risk neuroblastic tumours. Tumour angiogenesis offers a uniquely attractive therapeutic target. Vascular endothelial growth factor (VEGF), one of the most important angiogenic factors, is not only specific for the vasculature, but also plays a role in tumour cell survival and motility. Agents blocking VEGF signalling pathways have shown promising results in clinical trials against various tumours.

Microparticles (MPs) are a heterogeneous population of membrane-coated vesicles which can be released from virtually all cell types during activation or apoptosis¹. These particles serve as mediators of intercellular cross-talk and induce a variety of cellular responses. Our previous studies have demonstrated that lymphocyte-derived microparticles (LMPs) effectively inhibit endothelial cell proliferation, and potently suppress microvascularization in vitro and in an in vivo disease model of neovascularization through interfering with the VEGF signaling pathway¹⁸.

NB is a frequent pediatric tumor with a poor outcome in spite of aggressive treatment, and new therapeutic strategies are urgently needed. The current project introduces the challenging new concept that the biological message carried by LMPs could target both the tumour vasculature and the tumour cells themselves. We anticipate that our findings will provide new insights into the anti-cancer effect of LMPs in advanced NB and help for the development of more attractive therapeutic options.

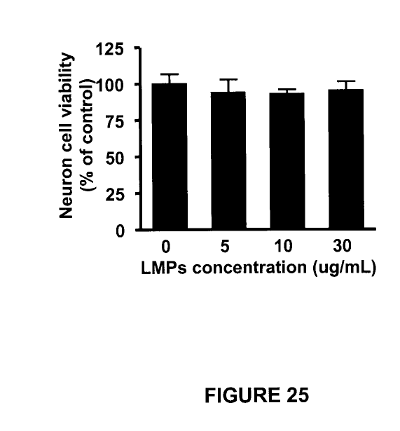

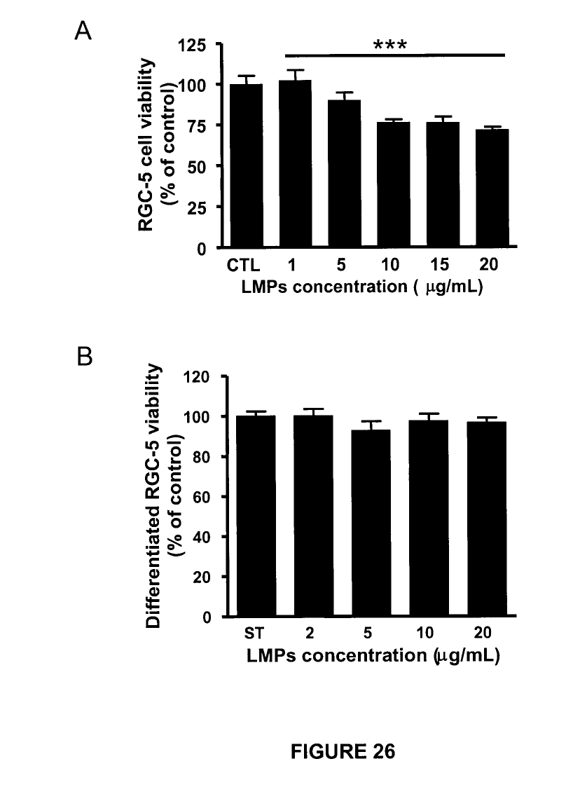

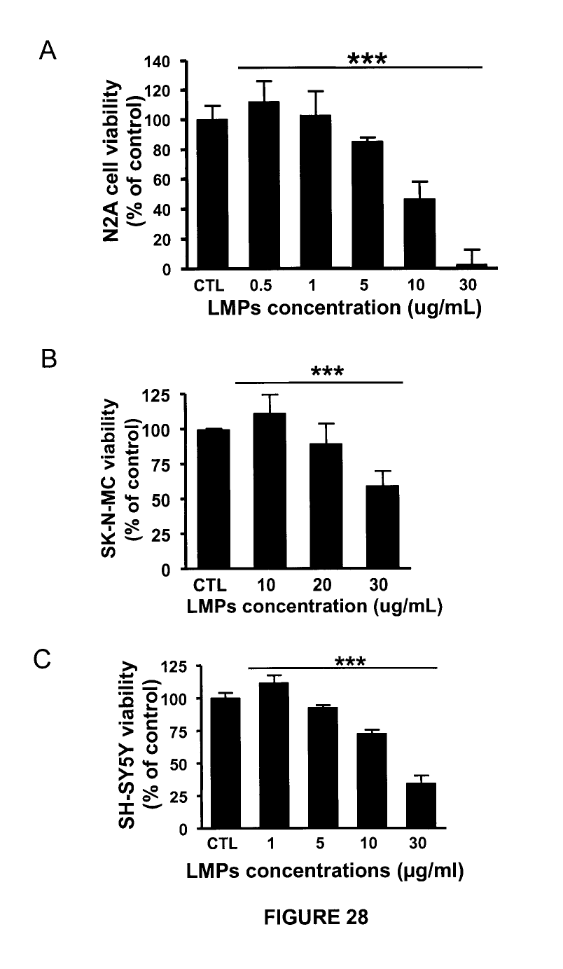

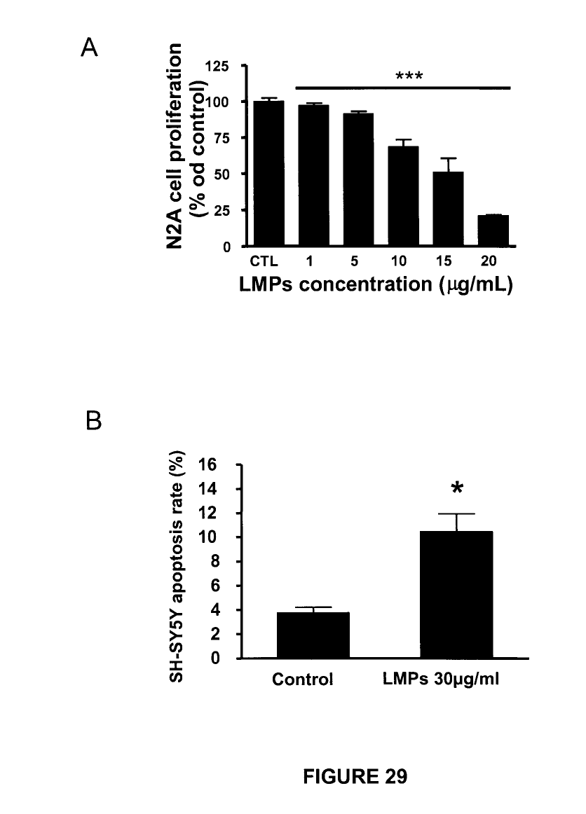

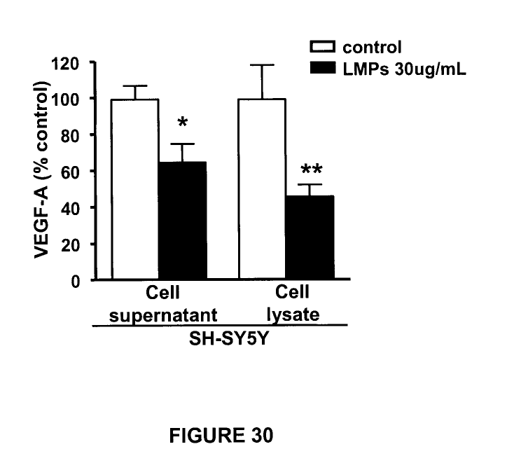

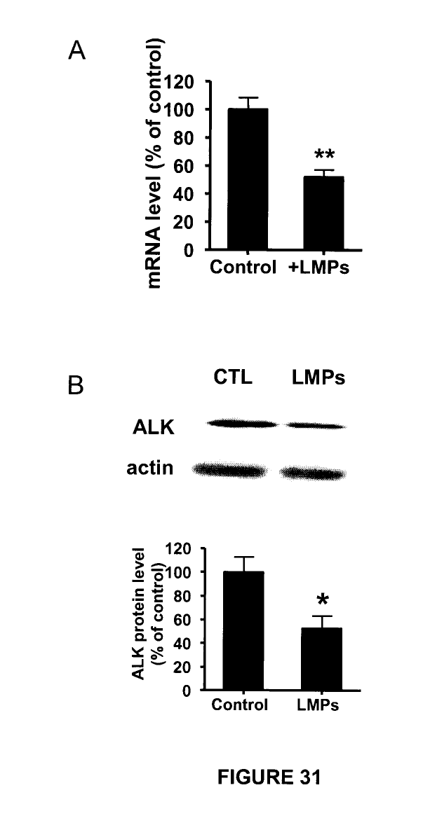

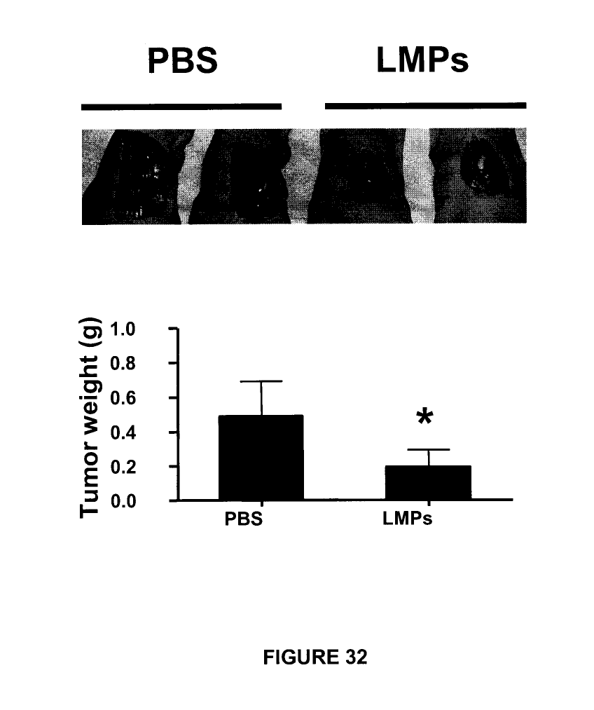

FIGS. 25-32 reveal the effect of LMPs on neuroblastoma (NB) cell viability. The results may be summarized as follows:
LMPs have no effect on confluent primary cultured adult rat cortical neurons (FIG. 25).
LMPs reduce high proliferating (RGC-5) viability in a dose-dependent manner; whereas they have no effect on the growth of terminal differentiated RGC-5 (FIGS. 26A,B).
LMPs reduce the viability of three different neuroblastoma (NB) cells (FIG. 28).
LMPs inhibit NB cell proliferation and induce human NB cells death (FIG. 29).
LMPs strongly down-regulate VEGF expression in human NB cells (FIG. 30).
LMPs inhibit NB growth through down regulating ALK expression (FIG. 31).
LMPs suppress human NB tumor growth in xenograft mice (FIG. 32).

### Example 4

Introduction:

Breast cancer is the most commonly diagnosed cancer and the 2nd leading cause of cancer death, in women in Canada. In 2008, an estimated 22,600 women will be diagnosed with breast cancer and 5,400 will die of it. One in 9 women is expected to develop breast cancer during her lifetime. Hormone-insensitive breast cancers represent approximately 30% of all breast cancers. They are unresponsive to antiestrogens, more likely to be poorly differentiated, of higher histological grade and are associated with a higher recurrence rate and decreased overall survival. There remains a significant unmet medical need to improve cancer-related clinical outcomes in hormone-insensitive breast cancer patients. http://caonline.amcancersoc.orq/cqi/content/full/54/3/150—R115-7#R115-7http://caonline.amcancersoc.orq/cgi/content/full/54/3/150—R117-7#R117-7http://caonline.amcancersoc.orq/cgi/content/full/54/3/150—R118-7#R118-7http://caonline.amcancersoc.orq/cgi/content/full/54/3/150—R119-7#R119-7The non-stopping proliferation and vigorous neovascularisation are two prominent characters of cancer. Anti-angiogenic agents are now being used successfully to treat several types of cancer and they predominantly act through inhibiting the vascular endothelial growth factor (VEGF) pathway. However, VEGF or VEGF receptor (VEGFR) inhibitors can have toxic effects on normal tissues, and tumours can develop resistance to these agents. There is a need to find other angiogenic factors that could be targeted to either circumvent resistance or reduce toxicity.

Lymphocytes-Derived Microparticles (LMP) are submicron vesicles shed from plasma membranes in response to cell activation, injury, and/or apoptosis. The measurement of the phospholipid content (mainly phosphatidylserine) of microparticles and the detection of proteins specific for these cells has allowed their quantification and characterization. Our laboratory has demonstrated that LMPs generated by apoptosis exert potent anti-angiogenic and anti-cancer effects. In vitro, LMPs inhibit the proliferation of several tumor cell lines and, can significantly reduce tumor size in vivo.

In summary, given the pivotal role of the uncontrolled cell growth and angiogenesis in metastatic breast cancer, understanding the mechanisms of how LMPs interrupt angiogenesis and breast cancer cells proliferation could provide attractive therapeutic strategies aimed at anti-metastatic breast cancer effects of LMPs.

The Anticancer Effect of LMPs on Breast Cancer

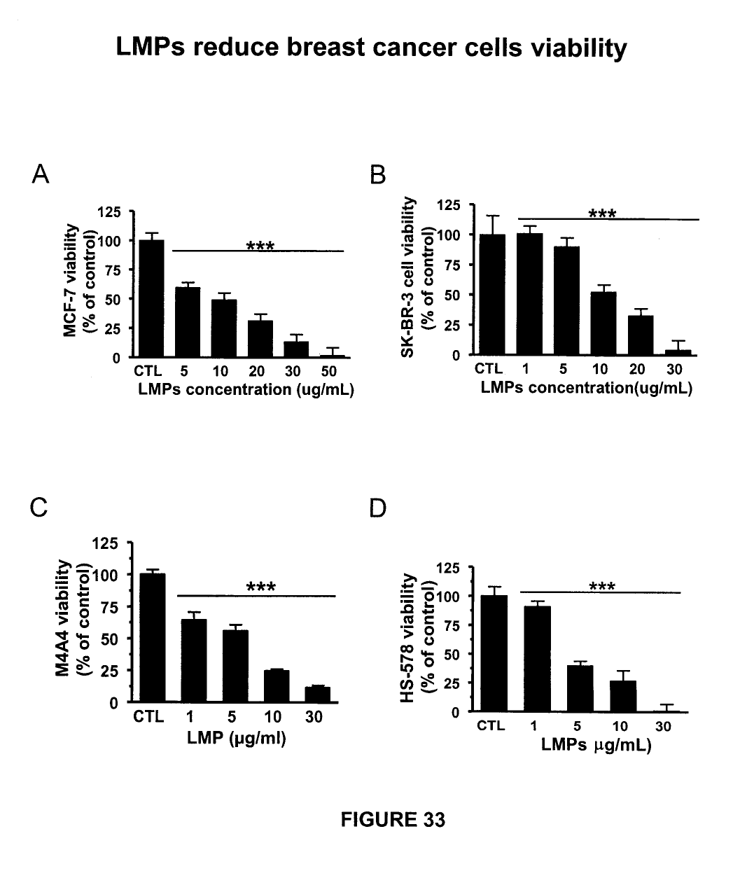

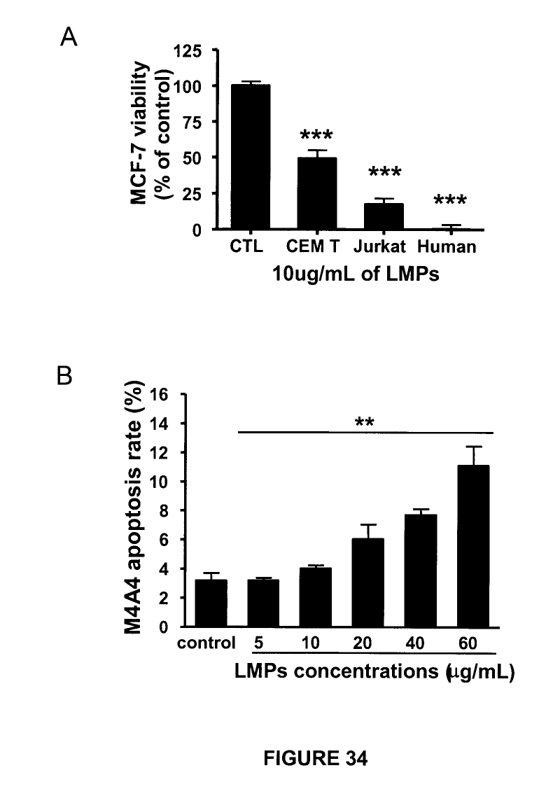

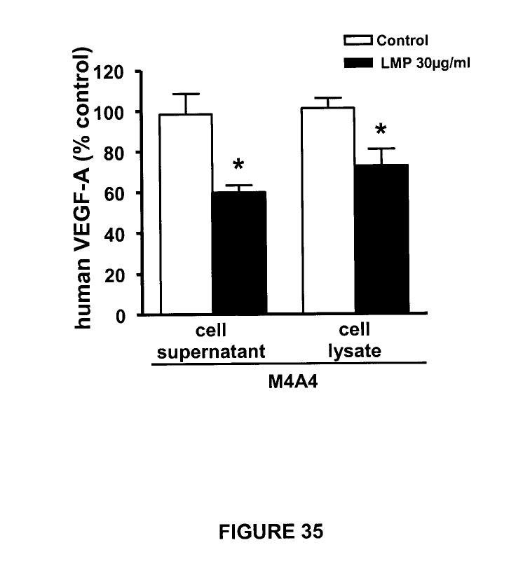

FIGS. 33-35 provide evidence that LMPs reduce breast cancer cell viability. In addition to the specific illustration of this in FIG. 33, FIG. 34 shows that LMPs derived from three different T lymphocyte sources reduce MCF-7 cell viability and dose-dependently induce breast cancer cells death. Morever, as revealed by the findings in FIG. 35, LMPs abrogate VEGF expression in human breast cancer cells (M4A4).

Although the present invention has been described hereinabove by way of preferred embodiments thereof, it can be modified without departing from the spirit, scope and nature of the subject invention, as defined in the appended claims.

### LIST OF REFERENCES

1. Hugel B, Martinez M C, Kunzelmann C, Freyssinet J M. Membrane microparticles: two sides of the coin. Physiology (Bethesda, Md. February 2005; 20:22-27.
2. Mallat Z, Benamer H, Hugel B, Benessiano J, Steg P G, Freyssinet J M, Tedgui A. Elevated levels of shed membrane microparticles with procoagulant potential in the peripheral circulating blood of patients with acute coronary syndromes. Circulation. Feb. 29 2000; 101(8):841-843.
3. Nieuwland R, Berckmans R J, Rotteveel-Eijkman R C, Maquelin K N, Roozendaal K J, Jansen P G, ten Have K, Eijsman L, Hack C E, Sturk A. Cell-derived microparticles generated in patients during cardiopulmonary bypass are highly procoagulant. Circulation. Nov. 18 1997; 96(10):3534-3541.
4. Tesse A, Martinez M C, Hugel B, Chalupsky K, Muller C D, Meziani F, Mitolo-Chieppa D, Freyssinet J M, Andriantsitohaina R. Upregulation of proinflammatory proteins through NF-kappaB pathway by shed membrane microparticles results in vascular hyporeactivity. Arteriosclerosis, thrombosis, and vascular biology. December 2005; 25(12):2522-2527.
5. Folkman J. Angiogenesis and apoptosis. Seminars in cancer biology. April 2003; 13(2):159-167.
6. Kim H K, Song K S, Chung J H, Lee K R, Lee S N. Platelet microparticles induce angiogenesis in vitro. British journal of haematology. February 2004; 124(3):376-384.
7. Mwaikambo B R, Sennlaub F, Ong H, Chemtob S, Hardy P. Activation of CD36 inhibits and induces regression of inflammatory corneal neovascularization. Investigative ophthalmology & visual science. October 2006; 47(10):4356-4364.
8. Sennlaub F, Valamanesh F, Vazquez-Tello A, El-Asrar A M, Checchin D, Brault S, Gobeil F, Beauchamp M H, Mwaikambo B, Courtois Y, Geboes K, Varma D R, Lachapelle P, Ong H, Behar-Cohen F, Chemtob S. Cyclooxygenase-2 in human and experimental ischemic proliferative retinopathy. Circulation. Jul. 15 2003; 108(2):198-204.
9. Canault M, Leroyer A S, Peiretti F, Leseche G, Tedgui A, Bonardo B, Alessi M C, Boulanger C M, Nalbone G. Microparticles of human atherosclerotic plaques enhance the shedding of the tumor necrosis factor-alpha converting enzyme/ADAM17 substrates, tumor necrosis factor and tumor necrosis factor receptor-1. The American journal of pathology. November 2007; 171(5):1713-1723.
10. Combes V, Simon A C, Grau G E, Arnoux D, Camoin L, Sabatier F, Mutin M, Sanmarco M, Sampol J, Dignat-George F. In vitro generation of endothelial microparticles and possible prothrombotic activity in patients with lupus anticoagulant. The Journal of clinical investigation. July 1999; 104(1):93-102.
11. Xu Q, Qaum T, Adamis A P. Sensitive blood-retinal barrier breakdown quantitation using Evans blue. Investigative ophthalmology & visual science. March 2001; 42(3):789-794.
12. Mezentsev A, Merks R M, O'Riordan E, Chen J, Mendelev N, Goligorsky M S, Brodsky S V. Endothelial microparticles affect angiogenesis in vitro: role of oxidative stress. Am J Physiol Heart Circ Physiol. September 2005; 289(3):H1106-1114.
13. Martinez M C, Tesse A, Zobairi F, Andriantsitohaina R. Shed membrane microparticles from circulating and vascular cells in regulating vascular function. Am J Physiol Heart Circ Physiol. March 2005; 288(3):H1004-1009.
14. Martin S, Tesse A, Hugel B, Martinez M C, Morel O, Freyssinet J M, Andriantsitohaina R. Shed membrane particles from T lymphocytes impair endothelial function and regulate endothelial protein expression. Circulation. Apr. 6, 2004; 109(13):1653-1659.
15. Cho S, Park E M, Febbraio M, Anrather J, Park L, Racchumi G, Silverstein R L, ladecola C. The class B scavenger receptor CD36 mediates free radical production and tissue injury in cerebral ischemia. J Neurosci. Mar. 9, 2005; 25(10):2504-2512.
16. Takahashi T, Yamaguchi S, Chida K, Shibuya M. A single autophosphorylation site on KDR/Flk-1 is essential for VEGF-A-dependent activation of PLC-gamma and DNA synthesis in vascular endothelial cells. The EMBO journal. Jun. 1, 2001; 20(11):2768-2778.
17. George R E, Sanda T, Hanna M, Frohling S, Luther W, 2nd, Zhang J, Ahn Y, Zhou W, London W B, McGrady P, Xue L, Zozulya S, Gregor V E, Webb T R, Gray N S, Gilliland D G, Diller L, Greulich H, Morris S W, Meyerson M, Look A T. Activating mutations in ALK provide a therapeutic target in neuroblastoma. Nature. Oct. 16, 2008; 455(7215):975-978.
18. Yang C, Mwaikambo B R, Zhu T, Gagnon C, Lafleur J, Seshadri S, Lachapelle P, Lavoie J C, Chemtob S, Hardy P. Lymphocytic microparticles inhibit angiogenesis by stimulating oxidative stress and negatively regulating VEGF-induced pathways. American journal of physiology. February 2008; 294(2):R467-476.

1. A method for the prevention or treatment of oxygen-induced retinopathy comprising administering lymphocytic-derived microparticles (LMPs) to a subject in need of such prevention or treatment.

2. The method as defined in claim 1, wherein said oxygen-induced retinopathy is retinopathy of prematurity.

3. A method for the prevention or treatment of cancer comprising administering lymphocytic-derived microparticles (LMPs) to a subject in need of such prevention or treatment.

4. The method as defined in claim 3, wherein said cancer is characterized by tumour development and progression.

5. The method as defined in claim 3, wherein said cancer is lung carcinoma, neuroblastoma, prostate cancer, cervical cancer, breast cancer, or cancer of the liver, colon or kidney.

6. A method for the prevention or treatment of a disease or process in which undesired angiogenesis occurs comprising administering lymphocytic-derived microparticles (LMPs) to a subject in need of such prevention or treatment.

7. The method as defined in claim 6, wherein said disease or process is selected from the following group:
ocular neovascular disease, such as retinal neovascularization, choroidal neovascularization, corneal neovascularization, corneal graft rejection, diabetic retinopathy, retinopathy of prematurity, macular degeneration, chronic uveitis/vitritis, scleritis, pemphigoid, corneal graft rejection, neovascular glaucoma, epidemic keratoconjunctivitis, infections causing retinitis or choroiditis, presumed ocular histoplasmosis, contact lens overwear, atopic keratitis, Terrien's marginal degeneration, marginal keratolysis, superior limbic keratitis, pterygium keratitis sicca, myopia, radial keratotomy, optic pits, chronic retinal detachment, hyperviscosity syndromes, trauma and post-laser complications associated with angiogenesis, rubeosis, and diseases caused by the abnormal proliferation of fibrovascular or fibrous tissue, Vitamin A deficiency, in syphilis, in Mycobacteria infections other than leprosy, in lipid degeneration, in chemical burns, in bacterial ulcers, in fungal ulcers, in Herpes simplex infections, in Herpes zoster infections, in protozoan infections, in Kaposi's sarcoma, in Mooren ulcer, in Terrien's marginal degeneration, in marginal keratolysis, by trauma, in rheumatoid arthritis, in systemic lupus, in polyarteritis, in Wegeners sarcoidosis, in Steven's Johnson disease, in sickle cell anemia, in sarcoid, in pseudoxanthoma elasticum, in Pagets disease, in Lyme's disease, in Eales disease, in Bechets disease, in hyperviscosity syndromes, in toxoplasmosis, in post-laser complications, in abnormal proliferation of fibrovascular tissue, in hemangiomas, in Osler-Weber-Rendu, in solid tumors, in blood borne tumors, in acquired immune deficiency syndrome, in osteoarthritis, by chronic inflammation, in Crohn's disease, in ulceritive colitis, in the tumors of rhabdomyosarcoma, in the tumors of retinoblastoma, in the tumors of Ewing sarcoma, in the tumors of neuroblastoma, in the tumors of osteosarcoma, in leukemia, in psoriasis, in atherosclerosis and in cancer.

8. A method to prevent human endothelial cell proliferation, migration and survival comprising administering lymphocytic-derived microparticles (LMPs) to a subject in need of such treatment.

9. The method as defined in claim 8, wherein said prevention occurs through modulation of the VEGF signal pathway.

10. The method as defined in claim 9, wherein said modulation of the VEGF signal pathway involves the downregulation of VEGFR2 and phosphorylated ERK1/2.

11. A method for inhibiting proliferation of cancer cells in a subject in need of such treatment comprising administering lymphocytic-derived microparticles (LMPs) to said subject.

12. The method as defined in claim 11, wherein said cancer is lung carcinoma, neuroblastoma, prostate cancer, cervical cancer, breast cancer, or cancer of the liver, colon or kidney.

13. The method as defined in any one of claim 1, 3 6, 8 or 11, wherein said LMPs are administered through a transfusion.

14. (canceled)

15. A method for treating or repressing the growth of tumours in a subject in need of such treatment comprising administration of lymphocytic-derived microparticles (LMPs) to said subject.

16. The method as defined in claim 15, wherein said LMPs are administered through a transfusion.

17. A pharmaceutical formulation comprising lymphocytic-derived microparticles (LMPs).

18.-23. (canceled)

24. A kit comprising lymphocytic-derived microparticles (LMPs).

25.-31. (canceled)

32. The method as defined in any one of claim 1, 3, 6, 8, 11 or 15, wherein said LMPs are derived from CEM T, Jurkat cells or T lymphocytes from human or animal peripheral blood.

33. (canceled)

34. A formulation as defined in claim 17, wherein said LMPs are derived from CEM T, Jurkat cells or T lymphocytes from human or animal peripheral blood.

35. A kit as defined in claim 24, wherein said LMPs are derived from CEM T, Jurkat cells or and T lymphocytes from human or animal peripheral blood.

36. A method of producing lymphocyte-derived microparticles (LMPs), said method comprising generating immortalized human or animal T lymphocyte cell lines from which the LMPs may be derived, and obtaining said LMPs from said cell lines.
